(19) United States
(12) Patent Application Publication
(10) Pub. No.: US 2022/0280074 A1
(43) Pub. Date: Sep. 8, 2022

(54) BEHAVIOR DETERMINATION APPARATUS, BEHAVIOR DETERMINATION SYSTEM, BEHAVIOR DETERMINATION METHOD, AND COMPUTER-READABLE STORAGE MEDIUM

(71) Applicant: Ochanomizu University, Tokyo (JP)

(72) Inventors: Yuji OHTA, Tokyo (JP); Julien TRIPETTE, Tokyo (JP); Nathanael AUBERT-KATO, Tokyo (JP); Dian REN, Tokyo (JP)

(21) Appl. No.: 17/664,945

(22) Filed: May 25, 2022

Related U.S. Application Data
(63) Continuation of application No. PCT/JP2019/046859, filed on Nov. 29, 2019.

(51) Int. Cl. A61B 5/11; A61B 5/103; A61B 5/00

(52) U.S. Cl. CPC A61B 5/1123; A61B 5/1038; A61B 5/7264

(57) ABSTRACT

A behavior determination apparatus includes a classification model to be used for classifying a behavior of a user, the behavior determination. The behavior determination apparatus includes a measurement data receiving device configured to acquire measurement data indicating a pressure or a force measured by one or more sensors provided on a sole surface of the foot of the user, a memory, and a processor configured to calculate a data feature by performing data processing on the measurement data and determine the behavior of the user by using the classification model.

### CROSS-REFERENCE TO RELATED APPLICATIONS

This application is a continuation application of International Application No. PCT/JP2019/046859 filed on Nov. 29, 2019, and designated the U.S., the entire contents of which are incorporated herein by reference.

### BACKGROUND OF THE INVENTION

### 1. Field of the Invention

The present invention relates to a behavior determination apparatus, a behavior determination system, a behavior determination method, and a computer-readable storage medium.

### 2. Description of the Related Art

Internet of Things (IoT) techniques are known. Further, methods of analyzing behavior of users in daily life are known by using IoT techniques.

Specifically, for example, a behavior determination apparatus first generates time-series data in which acceleration is measured by a sensor such as an acceleration sensor. The behavior determination apparatus then uses a time window to cut data from the time-series data. Further, the behavior determination apparatus calculates a plurality of feature values from time-series data, changing the size of the time window. The feature values are statistics such as mean or variance, Fast Fourier Transform (FFT) power spectrum, or the like. The behavior determination apparatus determines individual behaviors by assuming behaviors such as stopping, running, and walking, based on the feature values. When such individual behaviors can be determined, a method is known in which it is possible to judge the behavior as a whole and to determine the behavior with high accuracy, (for example, see Patent Document 1).

For example, a behavior determination apparatus acquires sensor data indicating acceleration or the like by communication. The sensor data is measured by an acceleration sensor or the like worn by the user or carried by the user. Subsequently, the behavior determination apparatus uses a determination model such as a neural network, a Support Vector Machine (SVM), a Bayesian network, or a decision tree to classify the behavior performed by the user into one of stopping, walking, running, going up and down stairs, getting on a train, getting in a car, riding a bicycle, and the like. Further, after the behavior is determined, the time interval until the next determination process is started is calculated, and the behavior determination apparatus performs the next determination process when the calculated time elapses. A method of reducing power consumption in such a way is known (for example, see Patent Document 2 and the like).

However, the conventional method may not be able to accurately determine the behavior of the user.

Accordingly, it is one object of the embodiments of the present invention to accurately determine the behavior of the user.

### RELATED-ART DOCUMENTS

### Patent Documents

Patent Document 1: Japanese Laid-Open Patent Publication No. 2011-120684
Patent Document 2: WO2013/157332

### SUMMARY OF THE INVENTION

According to one aspect of the embodiments, a behavior determination apparatus includes a classification model to be used for classifying a behavior of a user. The behavior determination apparatus includes a measurement data receiving device configured to acquire measurement data indicating a pressure or a force measured by one or more sensors provided on a sole surface of the foot of the user, a memory, and a processor configured to calculate a data feature by performing data processing on the measurement data and determine the behavior of the user by using the classification model.

### BRIEF DESCRIPTION OF THE DRAWINGS

FIG. 1 is a functional block diagram illustrating an example of a system configuration;

FIG. 2 is a diagram illustrating an example of data;

FIG. 3 is a diagram illustrating an example of data;

FIG. 4 is a diagram illustrating an example of data;

FIG. 5 is a diagram illustrating an example of data;

FIG. 6 is a diagram illustrating an example of data;

FIG. 7 is a diagram illustrating an example of a layout of a sensor position;

FIG. 8 is a block diagram illustrating an example of a hardware configuration related to information processing performed by an information processing device such as a measuring device, an information terminal, a server device, a management terminal, or the like;

FIG. 9 is a flow chart illustrating an example of an overall process;

FIG. 10 is a diagram illustrating an example of a decision tree;

FIG. 11 is a diagram illustrating a training data set used for a learning process and an example of a learning process;

FIG. 12 is a diagram illustrating a first example of classifying a behavior of a user;

FIG. 13 is a diagram illustrating a second example of classifying the behavior of the user;

FIG. 14 is a diagram illustrating an example of window acquisition;

FIG. 15 is a diagram illustrating an example of parameters;

FIG. 16 is a diagram illustrating an example of parameters;

FIG. 17 is a diagram illustrating an example of parameters;

FIG. 18 is a diagram illustrating approximate shapes of soles of a left foot and a right foot and an example in which four sensors are arranged on a front foot portion and one sensor is arranged on a rear foot portion;

FIG. 19 is a diagram illustrating an example of time-series data;

FIG. 20 is a diagram illustrating an example of an addition result;

FIG. 21 is a diagram illustrating an example of an FFT result;

FIG. 22 is a diagram illustrating an extraction example of a fundamental frequency of 0 to 150 Hz;

FIG. 23 is a diagram illustrating an extraction example of a fundamental frequency of 30 to 150 Hz;

FIG. 24 is an example of data illustrating measurement results of both feet before filtering;

FIG. 25 is an example of measurement data after filtering;

FIG. 26 is a functional block diagram illustrating a functional configuration example of a behavior determination system;

FIG. 27 is a functional block diagram illustrating a modification of the functional configuration example of the behavior determination system;

FIG. 28 is a diagram illustrating an example of a determination process of arbitrary measurement data by the behavior determination system;

FIG. 29 is a diagram illustrating experimental results;

FIG. 30 is a diagram illustrating experimental results using an SVM classification model; and

FIG. 31 is a diagram illustrating experimental results using a decision tree classification model.

### DESCRIPTION OF THE PREFERRED EMBODIMENTS

Suitable embodiments of the present invention will be described in the following, with reference to the accompanying drawings.

<Example of System Configuration>

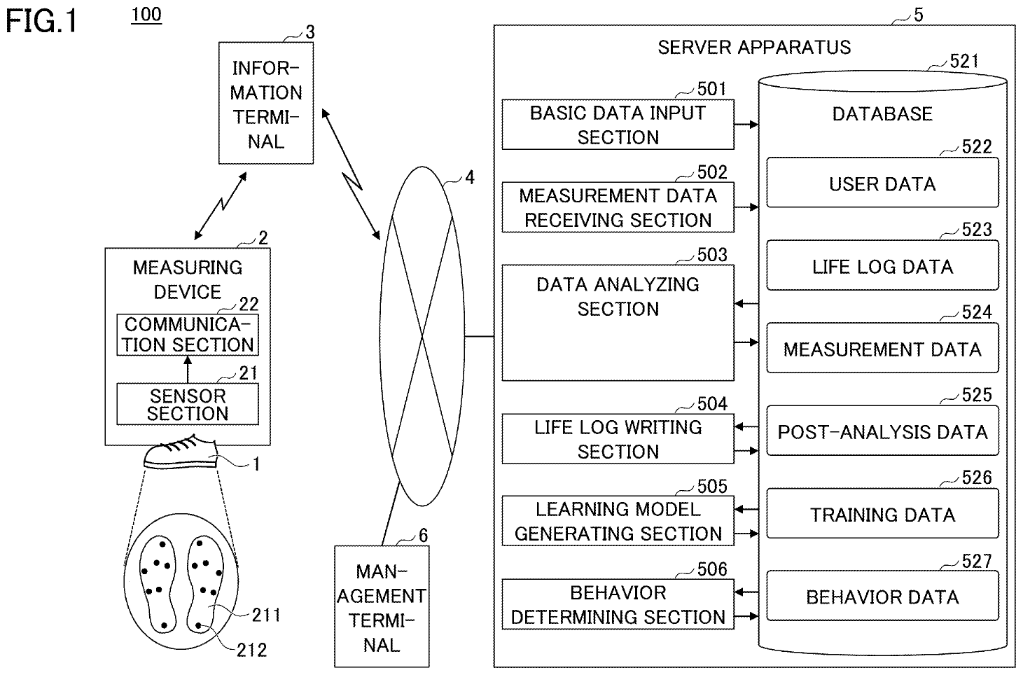

FIG. 1 is a functional block diagram illustrating an example of a system configuration. For example, a behavior determination system 100 includes a measuring device (an example illustrated below is an example of a shoe-shaped apparatus) 2, an information terminal 3, and a server device 5, or the like. The behavior determination system 100 may further include an information processing device, such as a management terminal 6 or the like, as illustrated in FIG. 1. In the following, the behavior determination system 100 illustrated in FIG. 1 will be described as an example. In the example of the behavior determination system 100 illustrated in FIG. 1, the server device 5 is a behavior determination device. Although the server device 5 is described as an example of the behavior determination device in the following, the behavior determination device may be used in a form other than that illustrated in FIG. 1.

In the behavior determination system 100, as illustrated in FIG. 1, the measuring device 2 is provided in a shoe 1 being used by a user (hereinafter, a left shoe and a right shoe are assumed to have the same configuration, and only one of the explanations is given; the shoes on the left and right form a pair).

As illustrated in FIG. 1, the measuring device 2 has a functional configuration including a sensor section 21 and a communication section (or device) 22.

First, the measuring device 2 measures pressure at a sole surface of the user's feet by the sensor section 21. Alternatively, the sensor section 21 may measure a force at the sole surface of the user's feet.

Next, the communication section 22 transmits measurement data measured by the sensor section 21 to the information terminal 3 by wireless communication such as Bluetooth (registered trademark), a wireless Local Area Network (LAN), or the like.

The information terminal 3 may be an information processing device, such as a smartphone, a tablet, a personal computer (PC), any combination thereof, or the like, for example.

The measuring device 2 transmits the measurement data to the information terminal 3 every 10 milliseconds (ms, or at 100 Hz), for example. In this manner, the measuring device 2 transmits the measurement data to the information terminal 3 at predetermined intervals set in advance.

The sensor section 21 may be formed by one or more pressure sensors 212 or the like, provided in a so-called insole type substrate 211 or the like, for example. The pressure sensor 212 is not limited to being provided in the insole. For example, the pressure sensor 212 may be provided in socks, shoe soles, or the like.

A sensor other than the pressure sensor 212, such as a shear force (frictional force) sensor, an acceleration sensor, a temperature sensor, a humidity sensor, any combination thereof, or the like, may be used in place of the pressure sensor 212.

Further, the insole may be provided with a mechanism for causing a color change (mechanism for providing visual stimulation), or a mechanism for causing material deformation or change in material hardness (mechanism for providing sensory stimulation), under a control from the information terminal 3.

The information terminal 3 may be provided with feedback of the state of the walking or feet to be indicated to the user. Moreover, the communication section 22 may transmit position data or the like, using a Global Positioning System (GPS) or the like. The position data may be acquired by the information terminal 3.

The information terminal 3 transmits the measurement data received from the measuring device 2 to the server device 5 via a network 4, such as the Internet, at predetermined intervals (for example, every 10 seconds or the like) set in advance.

In addition, the information terminal 3 may include functions such as acquiring data indicating a state of the user's walking, feet portion, or the like from the server device 5 and displaying the data on a screen, to feed back the state of the user's walking, foot portion, or the like, or to assist in the selection of shoes.

The measurement data or the like may be transmitted from the measuring device 2 directly to the server device 5. In this case, the information terminal 3 is used for performing operations with respect to the measuring device 2, making feedback to the user, or the like, for example.

The server device 5 has a functional configuration including a basic data input section 501, a measurement data receiving section 502, a data analyzing section 503, a behavior determining section 507, and a database 521, for example. The server device 5 may have a functional configuration including a life log writing section 504 or the like, as illustrated in FIG. 1. As an example, the server device 5 described in the following is assumed to have the functional configuration illustrated in FIG. 1, however, the server device 5 is not limited to the functional configuration illustrated in FIG. 1.

The basic data input section 501 performs a basic data input procedure for receiving (or accepting) basic data settings such as the user, the shoes, or the like. For example, the setting received by the basic data input section 501 is registered in user data 522 or the like of a database 521.

The measurement data receiving section 502 performs a measurement data receiving procedure for receiving the data or the like transmitted from the measuring device 2 via the information terminal 3. The measurement data receiving section 502 registers the received data in measurement data 524 or the like of the database 521.

The data analyzing section 503 performs a data analyzing procedure for analyzing the measurement data 524 and generating data after analyzing process 525 (hereinafter also referred to as “post-analysis data 525”) or the like.

The life log writing section 504 registers life log data 523 in the database 521.

A learning model generating section 505 performs a learning process based on the training data 526 or the like. In this manner, by performing the learning process, the learning model generating section 505 generates a learning model.

The behavior determining section 506 performs a behavior determining procedure for determining the user's behavior (including movement, action, or the like) by a behavior determining process or the like.

An administrator may access the server device 5 through the network 4 by the management terminal 6 or the like. The administrator may check the data managed by the server device 5, perform maintenance, or the like.

As illustrated in FIG. 1, the database 521 stores data including the user data 522, the life log data 523, the measurement data 524, the post-analysis data 525, the training data 526, the behavior data 527, or the like, for example. Each of these data may be configured as follows, for example.

<Example of Data>

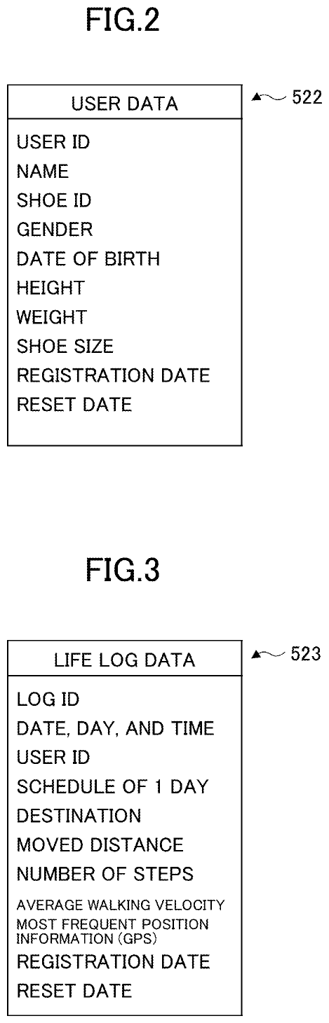

FIG. 2 is a diagram illustrating an example of the data.

The user data 522 includes items such as “user identification (ID)”, “name”, “shoe ID”, “gender”, “date of birth”, “height”, “weight”, “shoe size”, “registration date”, “update date”, or the like, as illustrated in FIG. 2. In other words, the user data 522 is the data for inputting features or the like of the user.

FIG. 3 is a diagram illustrating an example of the data.

The life log data 523 includes items such as “log ID”, “date, day, and time”, “user ID”, “schedule of 1 day”, “destination”, “moved distance”, “number of steps”, “average walking velocity”, “most frequent position information (GPS)”, “registration date”, “update date”, or the like, as illustrated in FIG. 3. In other words, the life log data 523 is the data indicating the user's behavior (which may include the schedule).

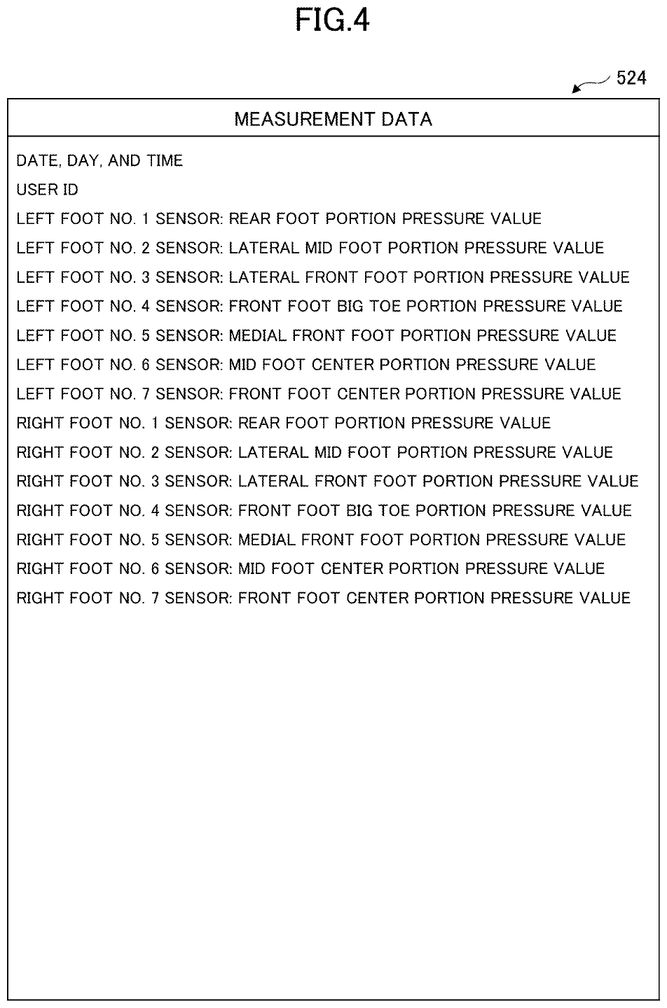

FIG. 4 is a diagram illustrating an example of the data.

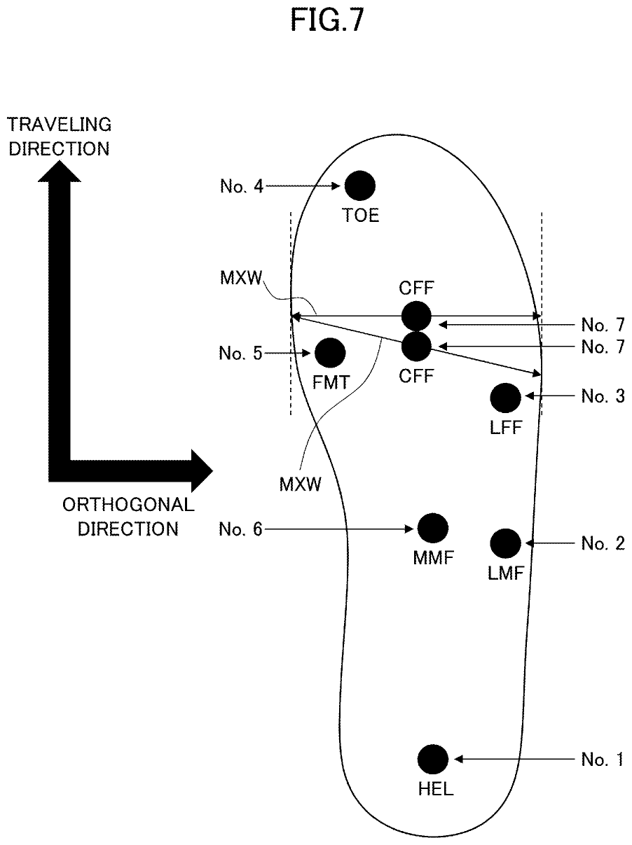

The measurement data 524 includes items such as “date, day, and time”, “user ID”, “left foot No. 1 sensor: rear foot portion pressure value”, “left foot No. 2 sensor: lateral mid foot portion pressure value”, “left foot No. 3 sensor: lateral front foot portion pressure value”, “left foot No. 4 sensor: front foot big toe portion pressure value”, “left foot No. 5 sensor: medial front foot portion pressure value”, “left foot No. 6 sensor: mid foot center portion pressure value”, “left foot No. 7 sensor: front foot center portion pressure value”, “right foot No. 1 sensor: rear foot portion pressure value”, “right foot No. 2 sensor: lateral mid foot portion pressure value”, “right foot No. 3 sensor: lateral front foot portion pressure value”, “right foot No. 4 sensor: front foot big toe portion pressure value”, “right foot No. 5 sensor: medial front foot portion pressure value”, “right foot No. 6 sensor: mid foot center portion pressure value”, “right foot No. 7 sensor: front foot center portion pressure value”, or the like, as illustrated in FIG. 4. An example of a specific layout of each sensor will be described later in conjunction with FIG. 7 or the like. In addition, each pressure value indicated by the measurement data 524 may have a format of waveform data plotted during measured time.

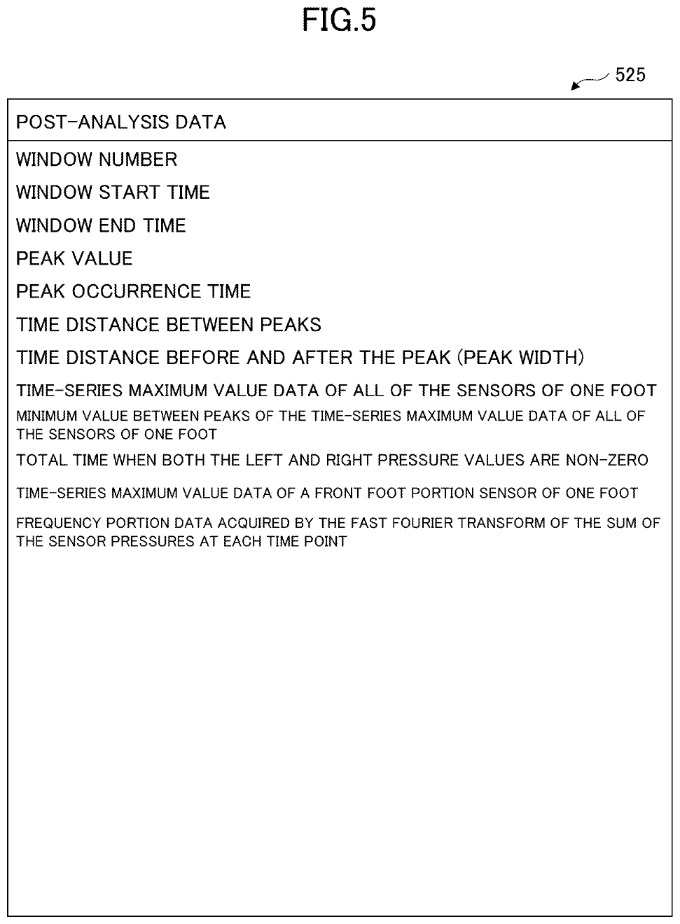

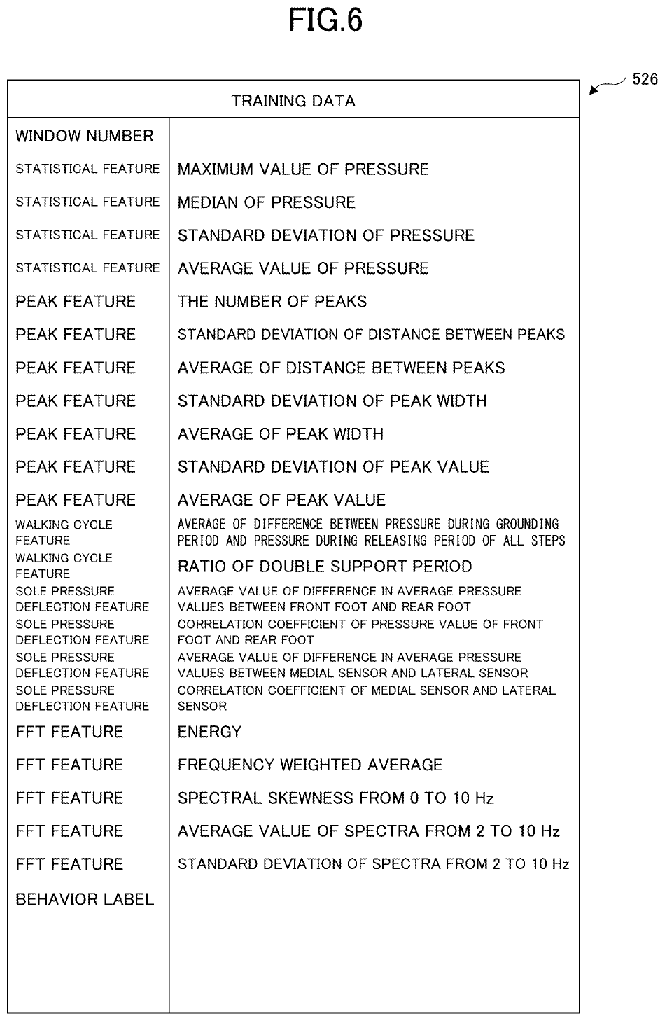

FIG. 5 and FIG. 6 are diagrams illustrating the example of the data.

The post-analysis data 525 is data representing the results of analyzing the measurement data and calculating a peak or the like as well as setting contents of a window or the like, as illustrated in FIG. 5.

When multiple windows exist, a “window number (window No.)” is a serial number or identification number for identifying each window.

A “window start time” indicates when the window starts.

A “window end time” indicates when the window ends.

A “peak value” is a value indicated by the peak point.

A “peak occurrence time” indicates a point at which the peak point was extracted.

A “time distance between peaks” indicates the average of the time interval from the time when the previous peak point was extracted to the time when the next peak point (which is the target peak point) occurred.

A “time distance before and after the peak (peak width)” indicates the time interval at which data indicating a predetermined value or more occurs before and after a certain peak point.

A “time-series maximum value data of all of the sensors of one foot” is time-series data that continuously stores the maximum value at each time point in the measurement data measured by all of the sensors of one foot in chronological order.

A “minimum value between peaks of the time-series maximum value data of all of the sensors of one foot” indicates the minimum value between the peak point and the next peak point indicated by the “time-series maximum value data of all of the sensors of one foot”.

A “total time when both the left and right pressure values are non-zero” indicates the sum of the times when neither the pressure of the left foot nor the right foot is “0” (that is, the foot touches the ground and pressure is generated).

A “time-series maximum value data of a front foot portion sensor of one foot” is time-series data that continuously stores the maximum value at each time point in the measurement data measured by the front foot portion sensor among all of the sensors in chronological order.

A “frequency portion data acquired by the fast Fourier transform of the sum of the sensor pressures at each time point” is data indicating the result of the fast Fourier transform (FFT) performed on the time-series data acquired by summing up the measured values indicated by all of the sensors at each time point.

The training data 526 is data indicating such as “window number”, “statistical feature”, “peak feature”, “walking cycle feature”, “sole pressure tendency feature”, “FFT feature”, “behavior label”, as illustrated in FIG. 6.

The “window number” is the same data as the post-analysis data 525.

The “statistical feature” is a value acquired by statistical processing of pressure values, such as maximum, median, mean, and standard deviation.

The “peak feature” includes the number of peak points, the interval between peak points (including values acquired by statistical processing such as mean and standard deviation), the width of the peak (including values acquired by statistical processing such as mean and standard deviation), and the value of the peak point (including values acquired by statistical processing such as mean and standard deviation).

The “walking cycle feature” is a value acquired by analyzing leg phase data or the like indicating steps of walking.

The “sole pressure tendency feature” is a value acquired by analyzing how the pressure applied to the sole surface of the foot is biased in the anteroposterior direction and the medial-lateral direction.

The “FFT feature” is a value obtained from the processing result of performing FFT on the data obtained by summing up the pressure values measured by all of the sensors of one foot in chronological order. A detailed explanation of the “FFT feature” will be described below.

The “behavior label” indicates a predetermined category of the behavior of the user.

The behavior data 527 is data indicating the result of determining the behavior of the user by the behavior determining section 506. That is, the behavior data 527 includes what kind of behavior the user has demonstrated.

The user data 522 and the life log data 523 are not essential data. In addition, the measurement data 524, the post-analysis data 525, and the training data 526 are not required to be the data as illustrated in the drawings. Further, each data is not required to include items as illustrated in the drawings. That is, the measurement data 524 may be data representing a pressure or a force measured by the sensor section 21. Therefore, statistical values such as as mean, variance, standard deviation, median, or the like may be calculated and generated in a case of being used for the subsequent processing, and are not essential components.

The behavior determination system 100 is not required to be configured as a whole as illustrated. For example, the measuring device 2, the information terminal 3, the server device 5, and the management terminal 6 may be integrated.

However, in the system configuration, as illustrated in FIG. 1, it is preferable that a configuration for generating measurement data is installed in the shoe 1, such as the sensor section 21 and the communication section 22, and it is preferable that the server device 5 or the like for processing and storing the measurement data is installed separately from the shoe 1. Specifically, in the behavior determination system 100, it is preferable that a sensor and a transmitter for transmitting the measurement data, a receiver for receiving the measurement data, an arithmetic section for performing the processing based on the measurement data, and the like are separate devices connected via the network.

Since many sensors and communication devices are compact and light-weight, the behavior of the user is unlikely to be affected even if the sensors and communication devices are installed in the shoe 1. On the other hand, a device including the arithmetic section, a storage, or the like is a large-sized device compared to the sensors or the like, as in the case of the server device 5. Therefore, the server device 5 is preferably installed in a place such as a room in which the information processing device is managed.

Further, the device installed in the shoe 1 is susceptible to breaking by the user exercising heavily or by being used in a harsh environment such as rainy weather. Therefore, the hardware configuration in which the easy-to-replace hardware is installed in the shoe 1 is preferably used as the sensor section 21.

On the other hand, in many cases, when the hardware configuration is such that the sensors, electronic circuits, or the like are all installed in the shoe 1 (for example, the configuration illustrated in Japanese Patent Application Laid-Open Publication No. 2009-106545), even in a case where a part of the components breaks, such as when only the sensor breaks, all of the components, including the parts that can still be used, are required to be replaced.

Therefore, among the hardware used to implement the behavior determination system and the behavior determination apparatus, the shoe 1 is preferably provided with a hardware configuration in which hardware has characteristics such as low cost, small size, light weight, ease to replace, high durability against an impact or the like because the shoe 1 is in an environment where the hardware is susceptible to breaking.

<Example of Sensor Layout>

FIG. 7 is a diagram illustrating an example of a layout of sensor positions. For example, sensors may be positioned as illustrated. For example, as “No. 7 sensor”, the sensor is preferably installed in the center portion of the sole surface of the foot in the direction perpendicular to the user's traveling direction (i.e., the vertical direction in FIG. 7) or in the center portion of the widest width (any position on the line of “maximum width MXW” in FIG. 7) in the width of the shoe. Hereinafter, the direction perpendicular to the user's traveling direction is simply referred to as “orthogonal direction”. The orthogonal direction is the horizontal direction in FIG. 7. That is, the sensor position is the center of the line connecting the ends of the first metatarsal bone and the fifth metatarsal bone, or the center of the line connecting the ball of the big toe and the ball of the little toe.

Alternatively, the sensors at other positions may be omitted, or the sensors may be positioned at other locations than illustrated. However, the position of the sensor may not be precise to the illustrated position, for example, the position of the sensor may be calculated from the measurement data measured by other sensors.

A sensor layout preferably includes at least a sensor at the position illustrated in “No. 7 sensor”. With this sensor layout, the behavior determination system 100 can determine the behavior more accurately than, for example, Japanese Patent Application Laid-Open No. 2013-503660 which discloses a sensor measuring the big toe portion, the tip portion of the metatarsal bone, the portion in proximity to the edge of the foot, and the heel portion.

In addition, when the sensor is installed in pants or socks, the user is required to wear the pants or socks in which the sensor is installed. On the other hand, in the case of installing on the sole surface of the foot or the like as illustrated in FIG. 7, since only the insole or the like is dedicated, the behavior of the user can be determined with shoes that the user prefers by changing only the insole.

The output of the sensor is preferably not a binary output (i.e., the output is either “ON” or “OFF”) that indicates whether the foot is grounded or not, but a numerical output (the output indicating not only whether the foot is grounded but also the strength of force or pressure, such as by Pa). That is, the sensor is preferably a sensor capable of multi-stage or analog output.

With the binary output sensor, it is difficult to extract peak points or the like even when the measurement data is analyzed because the degree of the force or pressure is unknown. In addition, in the case of binary output, it may not be possible to calculate a value such as a statistical value of the average value or the maximum value. When the binary output sensor is used, the number of types that can be calculated is smaller than that when a numerical value or the like can be output. On the other hand, when a sensor that outputs the force or pressure as a numerical value is used, the behavior determination system 100 can accurately determine the behavior.

Further, the behavior determination system 100 can determine the behavior without being combined with a sensor that is installed in the pants or the like for measuring a tensile force or the like. That is, the behavior determination system 100 can determine the behavior without data on the angle of the user's knee joints. Accordingly, the behavior determination system 100 is a hardware configuration that eliminates the need for sensors for measuring knee joints or the like. The behavior determination system 100 is not a hardware configuration that combines multiple types of sensors, such as a Global Positioning System (GPS) (for example, a configuration as illustrated in Japanese Patent Application Laid-Open No. 2011-138530). The behavior determination system 100 is sufficient as long as sensors capable of measuring the force or pressure on the sole of the foot are provided.

In the layout example illustrated in FIG. 7, “No. 1 sensor” or the like measures the rear portion and generates the measurement data. In other words, the sensor provided at a rear foot portion HEL is an example of a sensor for measuring the rear portion at the sole surface. In addition, the sensor provided at the rear foot portion HEL is mainly targeted to measure a range called the “rear foot portion” which includes the heel or the like.

Further, in the layout example illustrated in FIG. 7, “No. 2 sensor”, “No. 6 sensor”, or the like measure the mid portion and generate the measurement data. In other words, the sensors provided at a lateral mid portion LMF and a mid foot center portion MMF are examples of the sensors for measuring the mid portion at the sole surface. In addition, the sensors provided at the lateral mid portion LMF and the mid foot center portion MMF are mainly targeted to measure a range called the “mid foot portion”.

Further, in the layout example illustrated in FIG. 7, the “No. 3 sensor”, “No. 4 sensor”, “No. 5 sensor”, “No. 7 sensor”, or the like measure the front portion and generate the measurement data. In other words, sensors provided at a lateral front foot portion LFF, a front foot big toe portion TOE, a medial front foot portion FMT, a front foot center portion CFF, or the like are examples of the sensors for measuring the front portion at the sole surface.

The sensors provided at the lateral front foot portion LFF, the front foot big toe portion TOE, the medial front foot portion FMT, and the front foot center portion CFF are mainly targeted to measure a range called the “front foot portion”.

<Example Hardware Configuration>

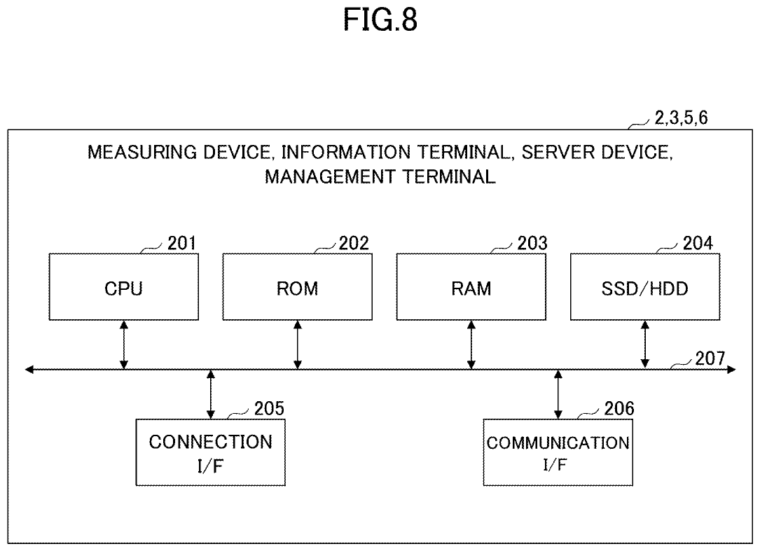

FIG. 8 is a block diagram illustrating an example of a hardware configuration related to information processing performed by an information processing device, such as a measuring device, an information terminal, a server device, a management terminal, or the like. As illustrated in FIG. 8, the information processing device, such as the measuring device, the information terminal, the server device, the management terminal, or the like is a general-purpose computer, for example. Hereinafter, an example will be described for a case where each information processing device has the same hardware configuration, however, each information processing device may have a different hardware configuration.

The measuring device 2 or the like includes a Central Processing Unit (CPU) 201, a Read Only Memory (ROM) 202, a Random Access Memory (RAM) 203, and a Solid State Drive (SSD)/Hard Disk Drive (HDD) 204 that are connected to each other via a bus 207. The ROM 202, the RAM 203, and the SSD/HDD 204 may form a computer-readable storage medium. In addition, the measuring device 2 or the like includes an input device and an output device, such as a connection interface (I/F) 205, a communication I/F 206, or the like.

The CPU 201 is an example of an arithmetic unit and a control unit. It is possible to perform each process and each control by executing a program stored in an auxiliary storage device, such as the ROM 202, the SSD/HDD 204, or the like, using a main storage device, such as the RAM 203 or the as a work area. Each function of the measuring device 2 or the like is implemented by executing a predetermined program in the CPU 201, for example. The program may be acquired through a computer-readable storage medium, acquired through a network or the like, or may be input in advance to the ROM 202, or the like.

According to the hardware configuration illustrated in FIG. 8, the measurement data receiving section 502, for example, may be formed by the connection I/F 205, the communication I/F 206, or the like. The data analyzing section 503 and the behavior determining section 506, for example, may be formed by the CPU 201, or the like.

<Example of Overall Process>

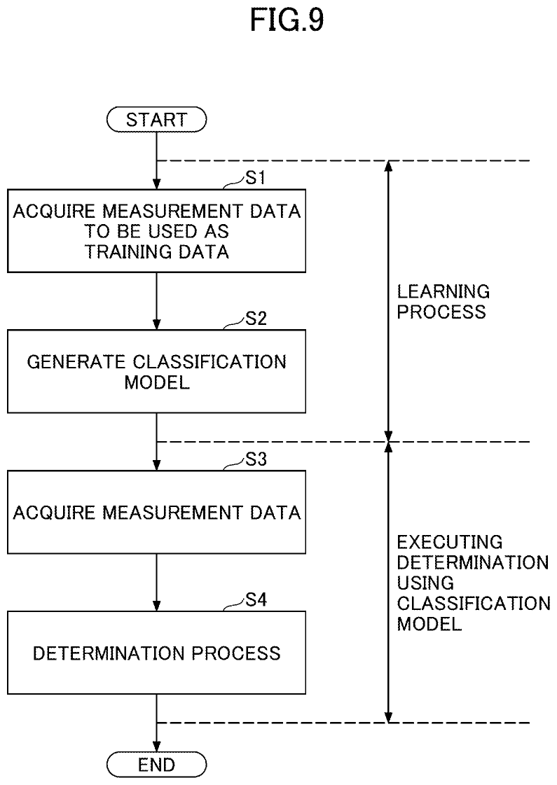

FIG. 9 is a flowchart illustrating an overall processing example. As illustrated in FIG. 9, the overall process includes a process of “learning process” which is a process of generating a model for classifying the behavior of the user (hereinafter referred to as “classification model”), and a process of “executing a determination using a classification model” based on the classification model that is generated in advance in the learning process.

Further, since the “learning process” and the “process of executing the determination using the classification model” may be performed only if the classification model is generated by the “learning process” before the determination using the classification model is performed, the process is not required to be executed continuously.

Alternatively, the overall process may be configured such that only executing the learning process to generate the classification model and then executing the determination using the classification model can be performed. That is, the classification model may have been generated at least once in advance, and the same classification model may be used multiple times, or the classification model may be generated for each determination using the classification model.

First, the learning process is performed in the order of step S1 and step S2, as illustrated, for example.

<Example of Acquiring Measurement Data as Training Data>

In step S1, the behavior determination apparatus acquires the measurement data that is to be used as the training data. The measurement data or the like are given a behavior label indicating the behavior taken when the measurement data is acquired.

<Example of Generating of Classification Model>

In step S2, the behavior determination apparatus generates a classification model.

The classification model is desired to be, for example, a decision tree as follows.

<Example of Classification Model>

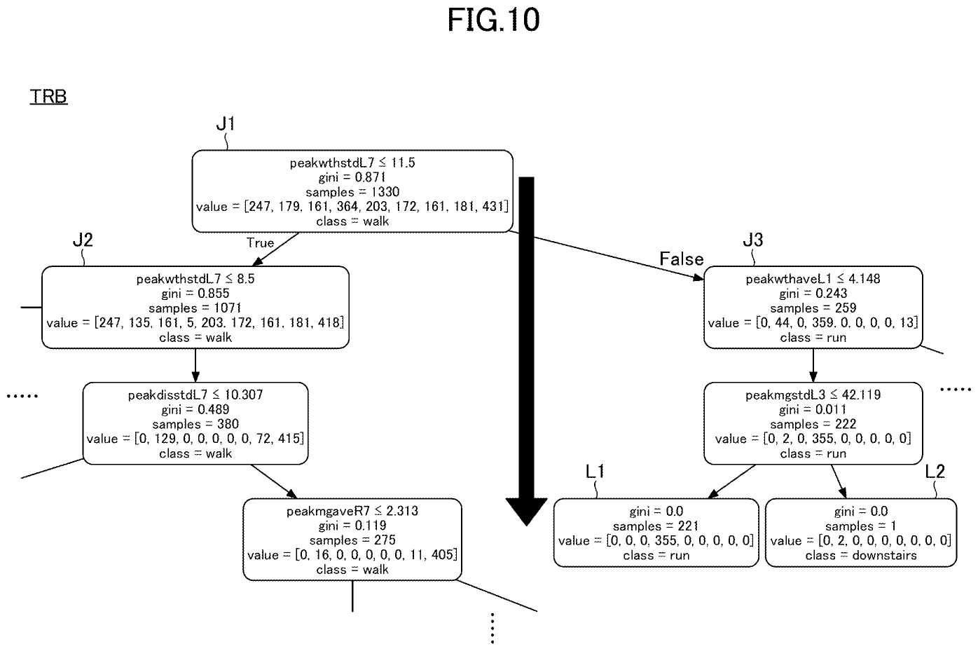

FIG. 10 is a diagram illustrating an example of a decision tree. The illustrated decision tree TRB is a part of the classification model generated by the learning process.

As illustrated in FIG. 10, the decision tree TRB is used to classify the behavior of the user indicated by the measurement data in the process of performing the determination using the classification model which is performed later. That is, in the learning process, several determinations are made in a stepwise manner and a determination process for ultimately classifying the behavior of the user is performed as in the decision tree TRB by using the training data acquired from the measurement data and the post-analysis data. Accordingly, the decision tree TRB is generated.

Specifically, when the measurement data is acquired in step S1, in the illustrated example, the post-analysis data 525 for acquiring the training data in the subsequent step is generated. The data feature acquired by the post-analysis data 525 is used as the training data, and the behavior determination apparatus performs the uppermost determination (hereinafter referred to as “first determination J1”). In other words, in the first determination J1, a determination condition with respect to the parameters (hereinafter simply referred to as the “determination condition”) is determined by performing learning processing of a value to be determined that is the training data (hereinafter referred to as “parameter”). When a plurality of determination conditions are determined in such a way, a classification model such as the decision tree TRB can be generated.

Hereinafter, a parameter will be used as an example of the data feature. The data feature is a value, a tendency, or the like that indicates the various features indicated by the measurement data. For example, the data feature is a parameter, such as a statistic value, which is calculated by performing a data processing, such as statistical processing, on the measurement data.

Note that the number of sensors may be one and the number of parameters may be one, and the total number of the data feature may be one. Alternatively, the number of sensors or parameters may be two or more, and the total number of the data feature may be plural.

Next, in the determination using the decision tree TRB illustrated in FIG. 10, if the training data satisfying the determination condition of the first determination J1 is a target (i.e., in FIG. 10, “True”), a determination with respect to a second determination J2 is performed.

On the other hand, if the training data that does not satisfy the determination conditions of the first determination J1 is the target (i.e., in FIG. 10, “False”), a determination with respect to a third determination J3 is performed. That is, the decision tree TRB performs the determination process in a stepwise manner (i.e., in FIG. 10, a plurality of determinations being made in a sequential manner from the top to the bottom) so that the determination of the second determination J2 or the third determination J3 is made next to the first determination J1. Accordingly, one determination result can be reached.

In FIG. 10, in each determination such as the first determination J1 to the third determination J3, the determination condition illustrated at the top (for example, “peakwthstdL” in the first determination J1 indicates the average of the peak widths in the left foot (represented by “L”)) indicates the data feature to which the determination condition is applied in the determination (in this case, the first determination J1).

Further, the other notation “gini” indicates Gini impurity. Further, the “samples” indicates the number of window records used in the determination. Further, the “value” indicates the number of processing of sample data. Further, the “class” indicates the behavior label given as a result of the determination. Note that other types may be included in the determination conditions.

Further, a plurality of decision trees TRBs are preferably generated. However, one decision tree TRB may be used. Thus, if there is more than one decision tree TRB in the one classification model, the behavior determination apparatus uses each decision tree TRB separately and performs the “process of executing the determination using the classification model” for each decision trees TRB.

The decision tree TRB is generated to have different determination conditions or parameters. Therefore, the “process of executing the determination using the classification model”, which is performed more than one time, often indicates different determination results (however, including cases in which all of the determination results are the same even under different determination conditions).

In this case, the classification model preferably collects the results of the determination by the decision tree TRB and performs the “process of executing the determination using the classification model” so as to adopt the most frequent determination results.

For example, training data indicating each feature of statistical feature, peak feature, walking cycle feature, FFT feature, sole pressure tendency feature, or a combination thereof is desired to be used as the data feature with regard to the parameter. Further, the parameter may be statistics such as the average of the plurality of these values. When such parameters are used, the behavior determination apparatus can accurately determine the behavior of the user. Details of the parameters are described later.

The classification model is not limited to the decision tree TRB illustrated in FIG. 10.

That is, the format of the classification model is not required to be a decision tree as long as the classification model is data that determines the determination conditions or the like that can classify the behavior of the user based on the parameters or the like based on the measurement data.

On the other hand, when a decision tree is included in the classification model, it is preferable that settings are made for the process of generating the decision tree, that is, the learning process. For example, if the settings are not made, “over-learning” (sometimes referred to as “overfitting” or the like) tends occur in the decision tree.

Therefore, in order to avoid the over-learning, in the learning process, it is preferable to consider in advance the number of decision trees included in the classification model (random forest, a forest where decision trees are gathered) and the minimum number of samples required to allow decision processing (branch of the tree).

By limiting the branch of the tree in the decision tree (mainly the number of branches in the decision tree), the maximum depth of the decision tree (in FIG. 10, the number of steps or boxes in the vertical direction) can be indirectly limited.

In addition, the minimum number of samples included at the end of the branch (for example, a first end L1 or a second end L2 in FIG. 10) may be set. For example, even if there is a number of samples necessary for branching by one upper determination, the branching is stopped when the number of samples is too small on one side of the next branch. In addition, the minimum value of the Gini impurity decreasing value (stopping branching if the branching does not substantially improve the “determination”) or the maximum value of the depth of the decision tree, or the like may be set.

When the learning process is performed, the type of parameters used in each determination, the values of parameters to be used in the criteria in the determination conditions, or a combination of thereof, are changed. Accordingly, the learning process may change the determination conditions and the values of parameters to be used in the determination. On the other hand, the determination conditions and the values of the parameters to be used in the determination may be set or changed by the user.

<Experimental Example of Over-Learning Mitigation Process>

For example, for each of the “10”, “20”, “100”, and “200” trees, the width of the “minimum number of samples required to allow branching” is set to “2” or “5”, and the learning process is repeated “10” times. The results of experiments in which “over-learning” was reduced under the above conditions are illustrated.

In this case, the set value of the “minimum number of samples required to allow branching”, which becomes the optimal “decision”, that is, the optimal value is “2” on average.

Next, the “minimum number of samples required to allow branching” is set to “2” with respect to a validation dataset and the “number of trees” is optimized in a more granular fashion.

Under the above conditions, the learning process was repeated “100” times for the “number of trees” from “1” to “500”. As a result of this experiment, “100” is the optimum experimental result for the “number of trees” of the decision tree in the classification model (random forest).

The results of the above experiments differ depending on the conditions under which the experiments are conducted. Thus, the optimum value set for the “minimum number of samples required to allow branching” and the “number of trees” are not limited to the above values.

<Example of Learning Process> (Training Phase)

In step S1 and step S2 described above, for example, a learning process is performed as follows.

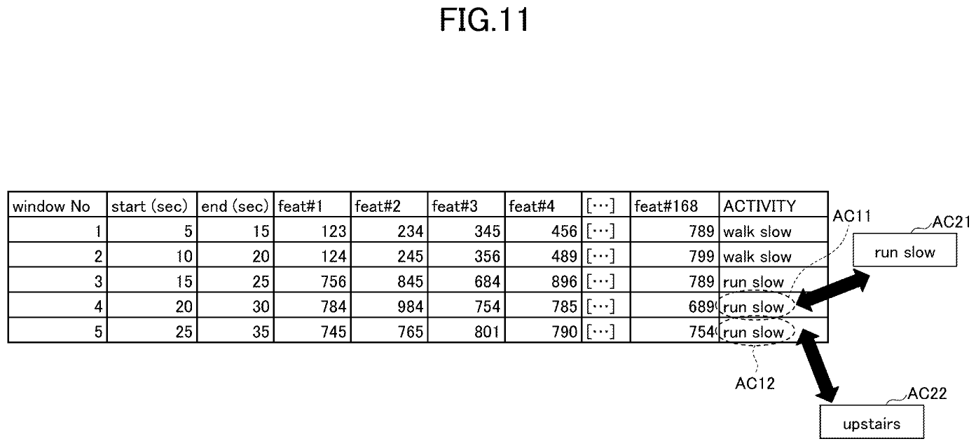

FIG. 11 is a diagram illustrating a training data set and a learning process example used in a learning process. In FIG. 11, the “window No” is a serial number used to identify the window (which will be described in detail below).

The “start(sec)” and “end(sec)” are values specifying a range of measurement data that becomes training data specified in the window, that is, a range of data used for learning. Specifically, the “start (sec)” indicates the start time of the window as the time elapsed from the start time of the measurement data (in this example, the system of units is “seconds”).

On the other hand, the “end(sec)” indicates the end time of the window as the time elapsed from the start time of the measurement data.

Specifically, when the “window No” is “1”, the data to be determined is the range of “10” seconds, where the start time is “5” seconds after the start time of the measurement data and the end time is “15” seconds after the start time of the measurement data.

The “feat #1” through “feat #168” are values calculated based on the measurement data 524 or the post-analysis data 525 and used in the determination as the data features. That is, the “feat #1” and the like indicate parameters. Therefore, this example is an example of calculating and determining different types of parameters of “168”. The number of parameters is not limited to “168”.

That is, the number of parameters is preferably determined based on the number of sensors or the location of the sensors (for example, the location of the sensor is only one foot or both feet, or the sensor is in the front foot portion or the rear foot portion, or the like).

When the number of sensors is increased, the number of parameters that can be generated based on the measurement data output by the sensors is often increased. Therefore, in order to use as many sensors as effectively as possible, it is preferable to increase or decrease the number of parameters according to the number of sensors.

The “ACTIVITY” indicates a behavior label given in advance for a behavior performed during the relevant window time. Therefore, in the learning process, the type of behavior actually performed by the user, that is, the “ACTIVITY” is correctly learned according to the condition of the data feature. In other words, learning is performed such that the type of behavior is classified according to the given behavior label.

Specifically, as in the case where the “window No” is “4”, the actual behavior illustrated in “ACTIVITY” (which is “run slow” in FIG. 10, hereinafter referred to as “first activity AC11”) may coincide with the classification result (which is “run slow” in FIG. 10, hereinafter referred to as “first classification result AC21”). Thus, when the “window No” is “4”, the first activity AC11 and the first classification result AC21 are examples illustrating the same type of behavior. In such cases, the determination is evaluated as a “correct answer”.

On the other hand, as in the case where the “window No” is “5”, the actual behavior illustrated in “ACTIVITY” (which is “run slow” in FIG. 10, hereinafter referred to as “second activity AC12”) may not match with the classification result (which is “upstairs” in FIG. 10, hereinafter referred to as “second classification result AC22”). Thus, when the “window No” is “5”, the second activity AC12 and the second classification result AC22 are examples illustrating a result of different types of behavior. In such cases, the determination is evaluated as an “incorrect answer”.

In this way, in the learning process, the number of “correct answers” increases, and the classification model that collects the decision tree and a plurality of decision trees becomes large. That is, the classification model is generated such that the behavior of the user can be accurately determined.

The classification model is preferably able to classify the behavior of the user, for example, as follows.

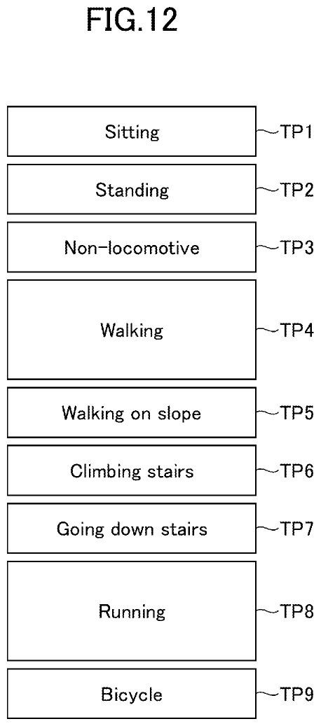

FIG. 12 is a diagram illustrating a first example of classifying the behavior of the user. As illustrated, the classification model is preferably able to ultimately label the behavior of the user into one of nine types. That is, the behavior label given in advance by “ACTIVITY” is preferably any of the nine types illustrated.

The “sitting” is a behavior label indicating that the user is sitting (hereinafter referred to as “sitting behavior TP1”).

The “standing” is a behavior label indicating that the user is standing (hereinafter referred to as “standing position behavior TP2”).

The “non-locomotive” is a behavior label indicating that the user is performing an action with no directivity in the direction of movement (hereinafter referred to as “non-locomotive behavior TP3”). An example of an action with no directivity is a household activity (such as vacuuming or drying laundry).

The “walking” is a behavior label indicating that the user is walking (hereinafter referred to as “walking behavior TP4”).

The “walking slope” is a behavior label indicating that the user is walking on an inclined walk (hereinafter referred to as “inclined walking behavior TP5”).

The “climbing stairs” is a behavior label indicating that the user is climbing the stairs (hereinafter referred to as “climbing stairs behavior TP6”).

The “going down stairs” is a behavior label indicating that the user is going down the stairs (hereinafter referred to as “going down stairs behavior TP7”).

The “running” is a behavior label indicating that that user is running (hereinafter referred to as “running behavior TP8”).

The “bicycle” is a behavior label indicating that the user is riding on a bicycle (hereinafter referred to as “bicycle behavior TP9”).

The type of behavior is preferably able to be further classified as follows.

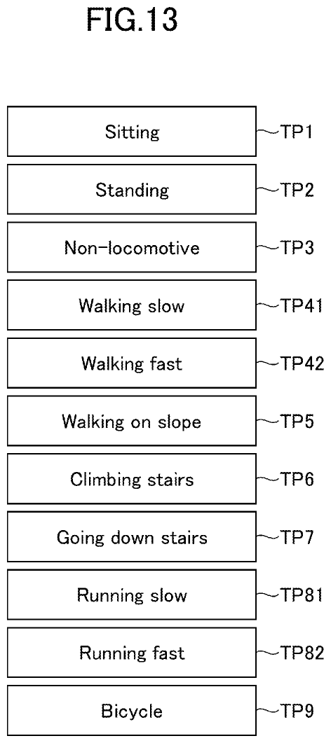

FIG. 13 is a diagram illustrating a second example of classifying the behavior of the user. Compared to the first example, the second example differs in that the behavior of the user is ultimately classified into one of 11 behavior labels, as illustrated. Specifically, the second example differs from the first example in that the walking behavior TP4 and the running behavior TP8 are further classified into two types. Hereinafter, the same points as the first example will be described with the same reference numerals and explanations will be omitted, focusing on different points.

The “walking slow” is a behavior label indicating that the user is walking at a low speed (hereinafter referred to as “slow walking behavior TP41”).

The “walking fast” is a behavior label indicating that the user is walking at a high speed (hereinafter referred to as “fast walking behavior TP42”).

The “running slow” is a behavior label indicating that the user is running at a low speed (hereinafter referred to as “slow running behavior TP81”).

The “running fast” is a behavior label indicating that the user is running at a high speed (hereinafter referred to as “fast running behavior TP82”).

Thus, the classification model preferably classifies the behavior such as running and the running is classified to be low speed or high speed. For example, settings such as allocating energy consumption per unit time may be performed in advance for each classified behavior. After performing the determination using the classification model, a process using the determination result may be performed in a later stage such as calculating the total energy consumption based on the type of the determined behavior.

As described above, when a subsequent process exists, it is more likely that the result of the subsequent process becomes more accurate when the behavior is classified in detail. Specifically, when calculating the total energy consumption, the total energy consumption can be calculated more accurately if the classification is finer as in the second example than in the first example.

<Verification Example> (Test Phase)

The data set of training data used in the learning process may also be used separately for learning and verification. For example, after generating a classification model with a training data set, it is determined to classify a validation data set with the generated classification model. When the data set is used separately for learning and verification, the data set used for verification does not include the “ACTIVITY” (i.e., the “behavior label”). Then, a determination is made to classify the verification data set by the classification model. After the determination, the correct “ACTIVITY” and the determination result are collated to verify the accuracy of the classification model.

In order to produce a more accurate classification model, the selection of the number and type of the data feature in performing the learning process is preferably manipulated. Further, the number of the data features is preferably between substantially “80” and “168”. In this case, it has been found to be more than about 80% accurate. The “statistical feature” and the “peak feature” are preferably selected preferentially as the type of the data features.

<Example of Performing Determination Using Classification Model>

After the learning process has been performed, that is, after the classification model has been prepared, the determination using the classification model is performed, for example, in step S3 and step S4.

<Example of Acquisition of Measurement Data>

In step S3, the behavior determination apparatus acquires the measurement data. The measurement data acquired in step S3 is not training data acquired in step S1, but the measurement data generated while the behavior of the user to be determined is being performed.

<Example of Determination Process>

In step S4, the behavior determination apparatus performs a determination process. The determination process preferably targets, for example, the following data determined by window acquisition. Alternatively, the determination process preferably uses the following parameters.

<Example of Acquiring Window>

A range of the measurement data to be determined is preferably determined by setting the window to slide along the time axis, for example, as follows. In this case, the measurement data of a single window leads to a data feature or the like constituting a single record of the data set for determination.

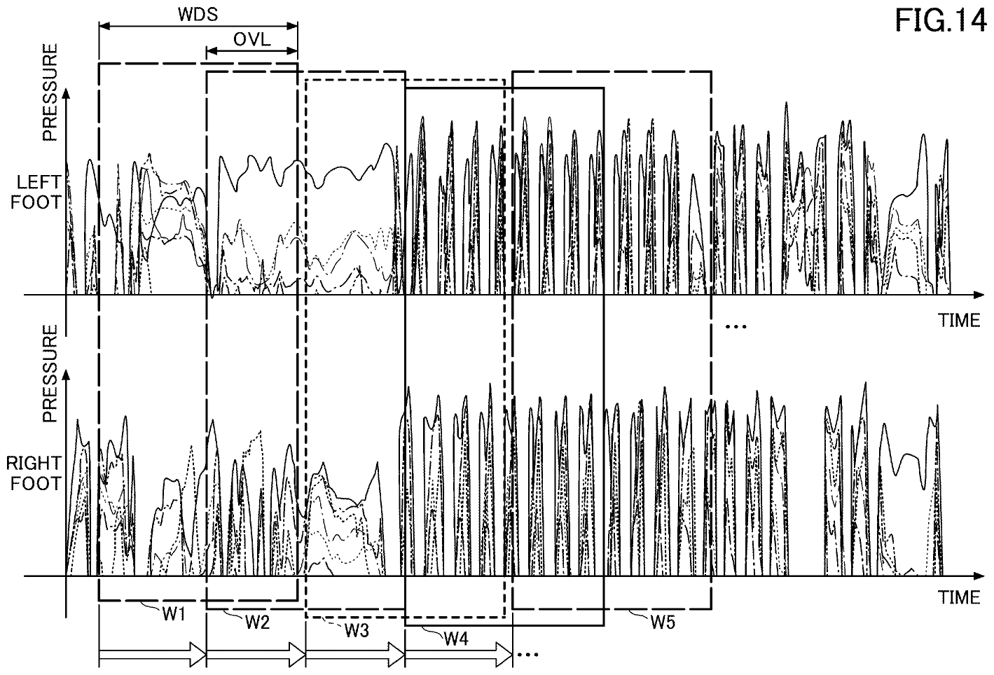

FIG. 14 is a diagram illustrating an example of window acquisition. In the illustrated example, the horizontal axis is the time axis and the vertical axis is the pressure. In addition, the upper figure is used as measurement data for the left foot, and the lower figure is used as measurement data for the right foot.

For example, as illustrated, the windows are set in the order of a first window W1, a second window W2, a third window W3, a fourth window W4, and a fifth window W5 (the windows are set to slide to the right in FIG. 14).

Further, the size of the window (hereinafter referred to as “size WDS”) can be set by the following Formula (1).

[Formula (1)]

windowsize=2^{ceil(log}^{₂}^{(2*f))} (1)

“windowsize” in Formula (1) refers to the time width of the data to be processed (the system of units is “seconds”). “f” is sampling frequency (the system of units is “Hz”). In addition, “ceil” is the number of data samples (the system of units is “pieces”).

It is preferable to slide the window, which is the size WDS of the value calculated by Formula (1), to acquire a plurality of ranges to be processed, such as the determination process, from a series of behavior measurement data.

However, the size of the window may be determined by taking into consideration the characteristics of the target user. For example, if the user has a characteristic of slow walking speed, the size of the window is preferably set large. That is, if there is a characteristic that one behavior is relatively slow, the size of the window may be set large so that the behavior is more likely to fit in the window.

Further, it is preferable that a plurality of windows are set so as to have different ranges, and that a part of the windows has a common range. Specifically, in the case of the first window W1 and the second window W2, the common range (hereinafter referred to as “overlapping portion OVL”) is preferably included in both, as illustrated in FIG. 14. Further, more than 50% of the window is preferably the common range. That is, the overlapping portion OVL preferably occupies more than 50% of the first window W1 and the second window W2. However, the overlapping portion OVL may be not limited to 50% or more but may be such as 25% to 75%.

The window preferably includes one cycle of a behavior. Without the overlapping portion OVL, if the time when the window is set once is in the middle of one cycle of the behavior, the data for one cycle is often not the target of analysis and learning. On the other hand, if the overlapping portion OVL exists, the target of the next window is started from the rear portion included in the previous window. Therefore, it is more likely that data that was not available for analysis in the previous window will be available in the next window.

Further, each window preferably has a change in the data pattern. If a single behavior continues for a predetermined period of time, the measurement data represents the same tendency over that period of time. As described above, when windows are taken from the measurement data of periodic data patterns, without the overlapping portion OVL, multiple windows often cut out the same data pattern and do not preserve diversity in analysis. On the other hand, if the overlapping portion OVL exists, the target of the next window is started from the rear portion included in the previous window. Therefore, there is a high possibility that the window can be cut with a data pattern different from that of the previous window. Accordingly, it is possible to increase the possibility that the behavior can be determined accurately.

However, if the overlapping portions OVL overlap too much, the same data will be determined many times, and the amount of calculation tends to increase. Therefore, the overlap portion OVL is preferably approximately 50%.

<Examples of Data Feature>

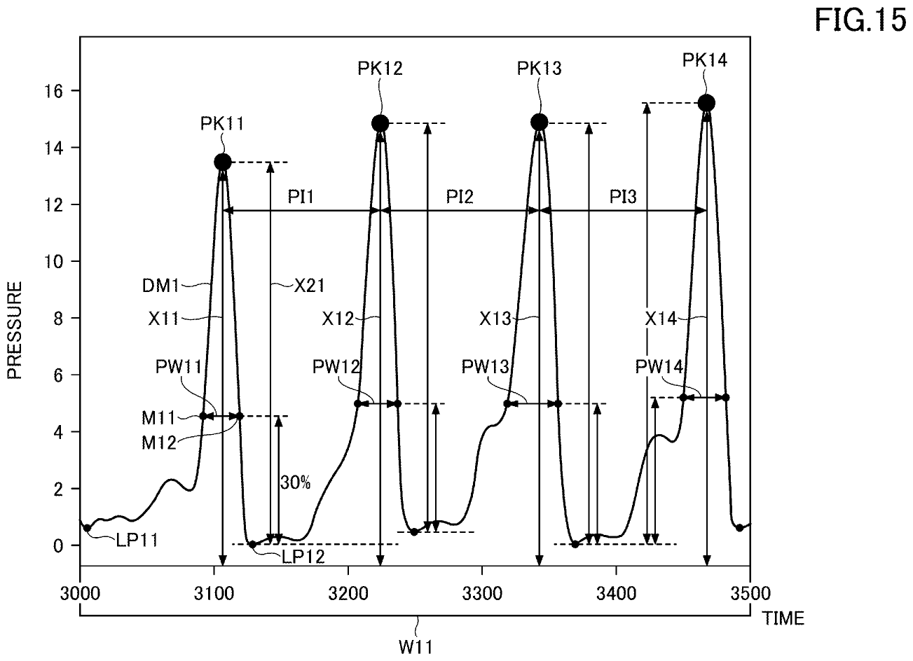

FIG. 15 is a diagram illustrating an example of parameters. FIG. 15 is an example of the measurement data measured by one sensor for one foot. In the illustrated example, the horizontal axis is the time axis and the vertical axis is the pressure (hereinafter, the example of pressure will be described, but force may be used). Hereinafter, an example will be described in which the measurement data illustrated in FIG. 15 (as a result of extraction in a window or the like) is the target of the determination. For example, the following parameters are preferably used in the determination process.

First, in the determination process, parameters are set for each window unit in which the measurement data is separated by a fixed time. Specifically, in FIG. 15, an 11th window W11 having a window start point of “3000 milliseconds” and a window end point of “3500 milliseconds” is set.

The parameters extracted in each window are values that indicate the results of specifying the so-called peak value and analyzing the height of the peak value, peak width, periodicity, or the like. The peak value may be the local maximum value or the maximum value of force or pressure in a predetermined division. The peak value may be extracted by differentiation or by a process such as specifying the highest value in comparison with other values.

In this case, the peak value used for analysis is extracted under the following conditions, for example. One is a condition in which the difference between the local maximum value and the minimum value after the local maximum value in the measurement data acquired from the same sensor is more than twice the standard deviation. Another one is a condition that the time difference between the local maximum value and the next local maximum value is 30 milliseconds or more. By using the peak value as the local maximum value that satisfies both of these conditions, the following parameters (i.e., data feature) can be extracted more accurately.

The data feature which becomes the parameter includes, for example, “statistical feature,” “peak feature”, “walking cycle feature”, “FFT feature”, and “sole pressure tendency feature”. All of these features are preferably used for learning, but at least one, or any combination, may be used.

<Example of “Statistical Feature”>

The parameters included in the statistical feature are, for example, the maximum pressure value, the median pressure value, the standard deviation of the pressure value, or the average pressure value. The statistical feature are also calculated from the measurement data for each sensor for every sensor measured in a window.

The maximum pressure value is the maximum value of the multiple local maximum values appearing in an 11th window W11 and is the maximum value of measured pressure data DM1 in the 11th window W11 (in this example, a value of a 14th peak point PK 14).

The median pressure value is the median value in the 11th window W11 of the measured pressure data DM1.

The standard deviation of the pressure value is the standard deviation in the 11th window W11 of the measured pressure data DM1.

The average pressure value is the average value in the 11th window W11 of the measured pressure data DM1.

<Example of “Peak Feature”≤

A first example of a parameter included in the peak features is, for example, the average of the peak values. Specifically, the average of the peak value is the value obtained by averaging the local maximum values or the maximum values specified as the peak value in the window, that is, the value obtained by summing up an 11th measured value X11, a 12th measured value X12, a 13th measured value X13, and a 14th measured value X14 and then dividing the obtained sum by “4”. That is, the average value of the local maximum value or the maximum value, such as the 11th measured value X11, the 12th measured value X12, the 13th measured value X13, and the 14th measured value X14 included in the measurement data, may be used as a parameter to determine the behavior.

In addition, the standard deviation of the peak value (including 3σ or the like) may be taken into consideration for parameters. When there is no specified peak value in the target window, the peak feature may be processed as “0 (zero)”.

A second example of the parameter included in the peak feature is the average of the intervals in the time axis of the peak points (hereinafter referred to as “peak intervals”). Specifically, the average value of the peak interval is a value acquired by adding a first peak interval PI1, a second peak interval PI2, and a third peak interval PI3 and dividing the total value by “3”. That is, the peak interval and the average value may be calculated from the value of the peak appearance time included in the data after the analysis process, or the average value may be calculated from the value of the time distance between peaks, and may be used as a parameter to determine the behavior.

In addition, the standard deviation of the peak interval (including 3σ or the like) may be taken into consideration for parameters.

Note that when only one peak value specified in the target window is present or no one peak value is detected, the peak feature may be processed as “0 (zero)”.

A third example of the parameter included in the peak feature is the time before and after the peak value at which the pressure is greater than a predetermined value. Hereinafter referred to as “peak width”.

Specifically, in order to calculate the peak width, first, the “height” is calculated centering on the target peak point. Then, at the time axis of an 11th peak point PK11, the previous occurrence of a previous minimum peak-to-peak value LP11 and the later occurrence of the subsequent minimum peak-to-peak value LP12 are compared to extract the smaller minimum peak-to-peak value. In this example, the minimum peak-to-peak value to be extracted is the minimum peak-to-peak value LP12.

Next, the difference between the extracted minimum peak-to-peak value LP12 and the 11th peak point PK11 (i.e., “height X21” in FIG. 15) is set to as a height.

Second, two points of a pressure value before the peak M11 and a pressure value after the peak M12 are specified. The pressure value M11 before the peak and the pressure value M12 after the peak are at the height position acquired by adding the value of “30%” of the height X21 to the extracted minimum peak-to-peak value LP12.

Third, a “first peak width PW11”, which is the width of the pressure value before the peak M11 and the pressure value after the peak M12 is calculated.

As a predetermined value, it is preferable to use a value at a position where the height is approximately “30%” from the minimum peak-to-peak value at which the value before and after the peak point is small, but the setting of the predetermined value is not limited to this.

In this example, the average value of the peak width is calculated for each peak in the window. That is, the average value of the peak width is obtained by summing up four values of a first peak width PW11, a second peak width PW12, a third peak width PW13, and a fourth peak width PW14 that appear in the 11th window W11, and then dividing the obtained sum by “4”. That is, the average value may be calculated from the value of the peak width included in the post-analysis data, and may be used as a parameter to determine the behavior. In addition, the standard deviation of the peak width (including 3σ or the like) may be taken into consideration for parameters. When there is no specified peak value in the target window, the peak feature may be processed as “0 (zero)”.

Another example of the parameter included in the peak feature includes the number of peaks. In this example, the 11th peak point PK11, the 12th peak point PK12, the 13th peak point PK13, and the 14th peak point PK14 are values calculated as “4” as the number in a 21st window W21. That is, the number may be calculated from the peak value or the value of the peak appearance time included in the post-analysis data, and may be used as a parameter to determine the behavior.

<Example of Walking Cycle Feature>

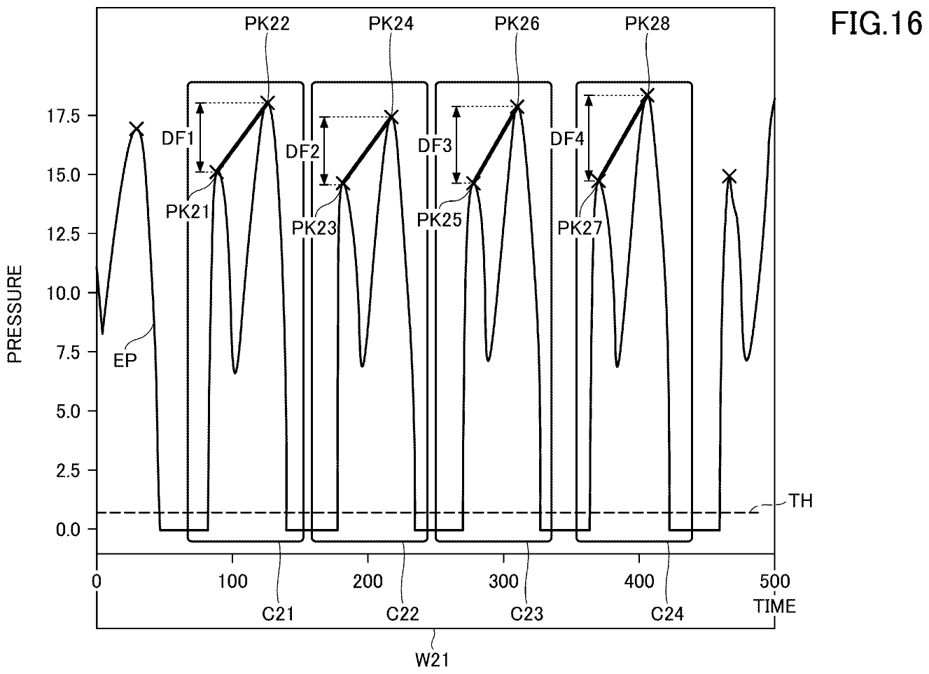

FIG. 16 is a diagram illustrating an example of parameters. FIG. 16 illustrates an example of data in which the maximum value is extracted for each time point from the measurement data measured by all the sensors installed on one foot and is continuous in time-series (i.e., time-series data EP after analysis). The horizontal axis is the time axis and the vertical axis is the pressure. A 21st window W21 having a window start point of “0 milliseconds” and a window end point of “500 milliseconds” is set. For example, in the determination process, it is preferable for the following parameters to be used.

In this example, the time-series data EP is divided into four cycles, for example, a 21st cycle C21, a 22nd cycle C22, a 23rd cycle C23, and a 24th cycle C24.

The examples described below are examples in which two peak points are calculated for each cycle of behavior, such as a 21st peak point PK21, a 22nd peak point PK22, a 23rd peak point PK23, a 24th peak point PK24, a 25th peak point PK25, a 26th peak point PK26, a 27th peak point PK27, and a 28th peak point PK28.

In this case, the behavior cycles are extracted by dividing the time-series data EP from the time period at “0 (zero)” to the next appearance of the time period at “0 (zero)”. The time-series data EP is the time-series in which the maximum value is extracted for each time point included in the post-analysis data measured by all the sensors installed on one foot. The behavior cycle corresponds to one step when applied to the mode of behavior.

In addition, in the time period where the time-series data EP is set to “0 (zero)”, the value “0” is not required as a reference. Specifically, as illustrated, a time point at which a threshold TH is set in advance and becomes less than the threshold TH may be used as a reference of “0 (zero)”. That is, a time point in which the force or the pressure is less than the threshold TH and becomes approximately “0”, or so-called “near zero”, may be used. In this case, for example, the threshold is set to “1”. However, the threshold may be other than “1”.

A first example of the parameter included in the walking cycle feature is the average value of the difference between two or more peak points included in a single window (hereinafter referred to as the “peak difference”). Specifically, in the 21st cycle C21, the peak difference is a first peak difference DF1.

The first peak difference DF1 is acquired by calculating the difference between the 21st peak point PK21 and the 22nd peak point PK22. The average value of the peak difference is a value acquired averaging a plurality of peak differences calculated for each cycle. That is, the average value of the peak difference is obtained by summing up the values of the first peak difference DF1, a second peak difference DF2, a third peak difference DF3, and a fourth peak difference DF4 and then dividing the obtained sum by “4”. In other words, from the time-series data EP representing the maximum value at the time point of all sensors of one foot included in the post-analysis data, the greatest local maximum value and the next greatest local maximum value are acquired for each cycle. Next, the average value of the plurality of peak differences calculated by using the peak difference between the two values may be used as a parameter to determine the behavior. In addition, the standard deviation of the peak difference (including 3σ or the like) may be taken into consideration for parameters.

If a cycle is not detected in the window, the walking cycle feature may be processed as “0 (zero)”. Further, even when two peaks are not detected in the cycle, the walking cycle feature may be processed as “0”.

The peak difference in this parameter corresponds to the difference between the pressure during the grounding period and the pressure during the releasing period, when applied to the behavior. That is, the 21st peak point PK21, the 23rd peak point PK23, the 25th peak point PK25, and the 27th peak point PK27 indicate the grounding period pressure of the foot in a certain behavior. On the other hand, the 22nd peak point PK22, the 24th peak point PK24, the 26th peak point PK26, and the 28th peak point PK28 indicate the releasing period pressure of the foot in a certain behavior. That is, the measurement data may be used as a parameter to determine the behavior. The parameter may be an average of the difference between the grounding period pressure and the releasing period pressure in all steps in the window.

A second example of the parameter included in the walking cycle feature is a ratio of double support period.

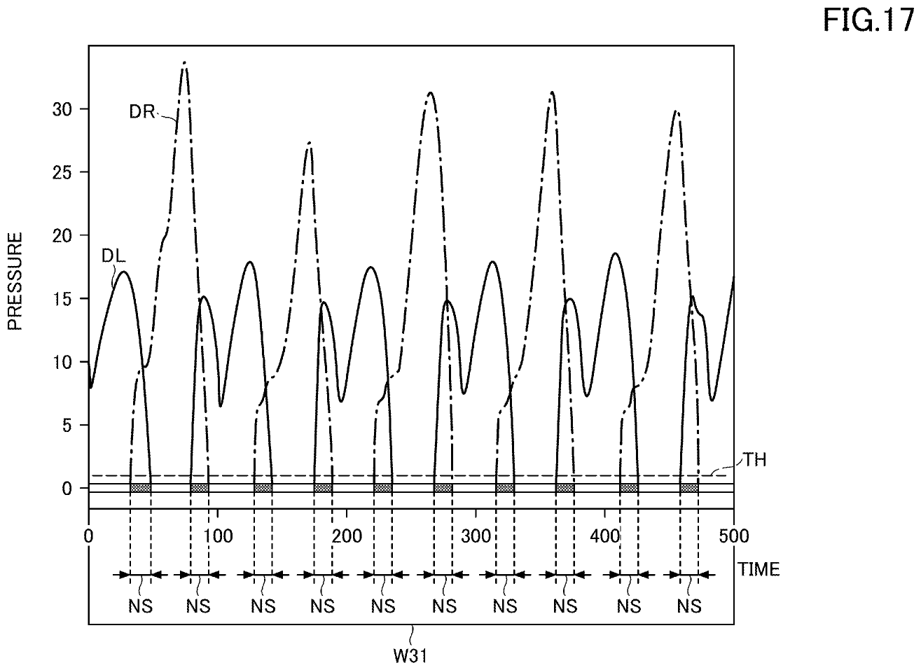

FIG. 17 is a diagram illustrating an example of parameters. FIG. 17 is a diagram illustrating an example of data representing both feet of the maximum value time-series data of all the sensors of one foot used in FIG. 16. FIG. 17 illustrates an example of data in which the maximum value is extracted from the measurement data for each time point from the measurement data by one or more sensors, being installed in each of the left foot and right foot, and is continuous in time-series. Similar to FIG. 15 or the like, the horizontal axis is the time axis and the vertical axis is the pressure (hereinafter, the example of pressure will be described, but force may be used).

A 31st window W31 having a window start point of “0 milliseconds” and a window end point of “500 milliseconds” is set.

In this example, as illustrated, the data illustrating the maximum value at the time point of all sensors for the left foot (hereinafter referred to as “left foot data DL”) and the data illustrating the maximum value at the time point of all sensors for the right foot (hereinafter referred to as “right foot data DR”) are displayed.

For example, the left foot, which is one of the left foot and right foot, is designated as a “first foot” and the right foot, which is the other foot, is designated as a “second foot”. In the example of FIG. 17, there is a point at which the first foot becomes “0” (hereinafter referred to as a “first time point”). On the other hand, in the example of FIG. 17, there is a point at which the second foot starts to increase from “0” (hereinafter referred to as a “second time point”). Next, if the first foot is the right foot and the second foot is the left foot, that is, the left foot and right foot are reversed, the first time point and the second time point are similarly generated.

In FIG. 17, the time between the first time point and second time point is illustrated by an interpoint NS. In the interpoint NS, the time period in which the pressure of both feet is not “0 (zero)” is called the “double support period”. In other words, the interpoint NS is a time period in which the pressure of the first foot decreases and becomes almost “0 (zero)”, that is, the first foot starts floating in the air away from the ground.

On the other hand, the interpoint NS is a time period in which the pressure of the second foot increases when the foot starts to touch the ground from the state where the pressure of the second foot almost “0 (zero)”. That is, a state where the second foot starts to touch the ground.

Therefore, the interpoint NS is a time period in which the grounding and non-grounding of both the left foot and right foot are switched, and the pressure of both feet can be detected.

The ratio of double support period is a value acquired by summing up the time widths of multiple interpoints NS and dividing the sum by the time width of the 31st window W31. That is, the sum of the times when the left and right pressure values included in the post-analysis data after are not “0 (zero)” may be used to calculate a parameter. The parameter may be the value obtained by calculating the ratio of time that the sum of the times occupies in the window. The parameter may be used to determine a behavior.

Note that “0” is not required to be a reference at the first and second time points. Specifically, as illustrated, the threshold TH may be set in advance, and the first time point and the second time point may be determined based on the time point below the threshold TH. In other words, by specifying a case in which the time-series data EP representing the maximum value at the point of all sensors of one foot included in the post-analysis data is below the threshold value, the first time point and the second time point, that is, the interpoint NS, may be calculated. That is, a time point in which the force or the pressure is less than the threshold TH and becomes approximately “0”, or so-called “near zero,” may be used. In this case, for example, the threshold is set to “1”. However, the threshold may be other than “1”.

Further, other than illustrated in the figure, a state in which one foot is in contact with the ground and the other foot is not in contact with the ground, that is, a time period where standing is maintained on one foot may be used.

For example, other than the so-called “double support period”, which is the time at which both the left foot and right foot are grounded, the time at which only one foot is grounded, that is, so-called “single support period” may be determined. The behavior may then be determined by, for example, the length of the single support period. For example, the length of the single support period, which is the length obtained by subtracting the total of interpoints NS from the time width of the 31st window W31, may be a parameter.

As described above, with respect to the walking cycle feature, synchronization of the left foot data DL and the right foot data DR may be used for the determination as parameters.

<Example of Sole Pressure Tendency Feature>

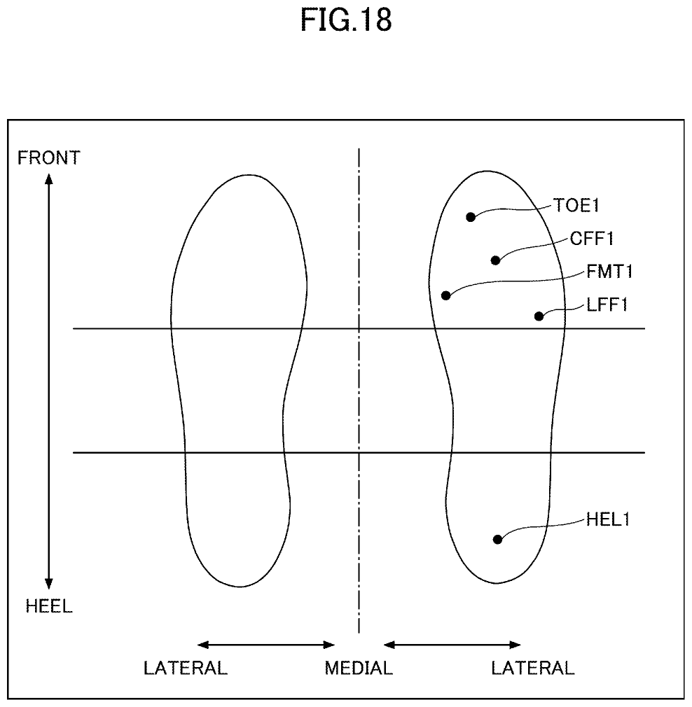

FIG. 18 is a diagram illustrating approximate shapes of soles of left foot and right foot and an example in which four sensors are arranged on a front foot portion and one sensor is arranged on a rear foot portion. For example, as illustrated, each foot is pre-divided into areas, such as a front portion, a center portion, a rear portion, a medial portion, and a lateral portion.

A first example of the parameter included in the sole pressure tendency feature is the average value between both feet for the difference in the average pressure values between the front foot and the rear foot, and the average value between both feet for the difference in the average pressure values between the medial and the lateral.

For example, the maximum value at each time point is extracted from the measurement data by multiple sensors installed in the front foot area (for example, sensors installed at a first front foot measurement point TOE1, a second front foot measurement point FMT1, a third front foot measurement point CFF1, and a fourth front foot measurement point LFF1) to acquire time-series data of the maximum value at the time point of the front foot sensor on one foot.

Further, the difference between the average of the time-series data of the maximum value at the time point of the front foot sensor on one foot and the average value of the sensor in the rear foot area (for example, the sensor installed in a rear foot measurement point HEL1) is acquired. Similarly, with regard to the opposite foot, the difference between the average of the time-series data of the maximum value at the time point of the front foot sensor on one foot and the average value of the sensor in the rear foot area is acquired. The behavior may be determined using the average value of both feet of this difference value as a parameter.

That is, the number of sensors installed in each area of the front foot portion and the rear foot portion compares the measured values of all or some of the sensors and uses the time-series data of the maximum value to calculate the average value. Meanwhile, when a single sensor is provided, the measured value of the sensor is used to calculate the average value.

A second example of the parameter included in the sole pressure tendency feature is a correlation function of the pressure values of the front foot and the rear foot and a correlation function of the pressure values of the medial and the lateral.

For example, it is preferable to use the Pearson correlation coefficient of pressure or force in the traveling direction (i.e., the direction of connecting the front foot and the rear foot) and the orthogonal direction (i.e., the direction of connecting the medial and the lateral) calculated by Formula (2) below.

$$\begin{matrix}[ Formula ( 2 ) ] & \\ r = \frac{{\sum }_{i = 1}^{n} ( {x}_{i} - \overline{x} ) ( {y}_{i} - \overline{y} )}{{( ( {\sum }_{i = 1}^{n} {( {x}_{i} - \overline{x} )}^{2} ) ( {\sum }_{i = 1}^{n} {( {y}_{i} - \overline{y} )}^{2} ) )}^{1 / 2}} & ( 2 )\end{matrix}$$

“r” in Formula (2) is the Pearson correlation coefficient. “x” and “y” in Formula (2) represent the values of the measured force or pressure in the traveling direction (vertical direction in FIG. 18) and the orthogonal direction (horizontal direction in FIG. 18). Accordingly, the index “i” of “x” and “y” in Formula (2) is a number for identifying each value. Therefore, if “i” is the same for “x” and “y”, the same measurement result is obtained, that is, the measurement is performed by the same sensor.

In Formula (2), “x” and “y” with an overline indicate the average value. “n” in Formula (2) is the number of data held by the measurement data.

For example, the correlation coefficient may be calculated by Formula (2) from the time-series data of the maximum value at the time point of the front foot sensor in one foot included in the post-analysis data, and the correlation coefficient may be used as a parameter to determine the behavior.

For example, the correlation coefficient may be calculated by Formula (2) based on the measurement data of the sensor located in the medial area (a second front foot measurement point FMT1 in FIG. 18) and the measurement data of the sensor located in the lateral area (the fourth front foot measurement point LFF1 in FIG. 18), and the correlation coefficient may be used as a parameter to determine the behavior. Such Pearson correlation coefficient can be used to make a more accurate determination.

The parameter included in the sole pressure tendency feature may include a pressure distribution or the like. That is, the behavior may be determined based on distribution such as an area of high pressure or an area of low pressure. The pressure may be an average value of the measurement data by the multiple sensors in the area.

<Example of FFT Feature>

For example, the following parameters are used for “FFT features” in FIG. 6.

The parameter included in the FFT feature is, for example, energy, frequency weighted average, spectral skewness from 0 to 10 Hz, average value of the spectra from 2 to 10 Hz, and standard deviation of the spectra from 2 to 10 Hz.

“FFTW” is frequency volume data obtained by the fast Fourier transform of the total sensor pressure values at each time point. That is, first, in the window, the sum of the pressure values at each time point of all sensors is calculated. Next, the frequency volume data acquired by the fast Fourier transform of the time-series data on the time axis becomes “FFTW”.

The second peak value that appears in the “FFTW”, the spectrum of the FFTW, standard deviation, power spectral density, entropy, or the like may be calculated to be used as a parameter to determine the behavior.

Specifically, the parameter of “FFT feature” is generated as follows.

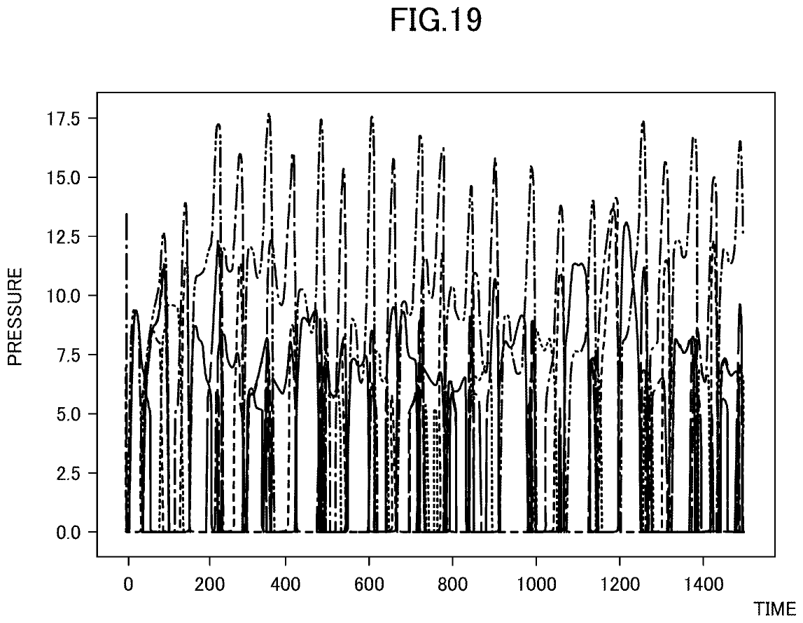

FIG. 19 is a diagram illustrating an example of the time-series data. Hereinafter, a case where seven sensors are installed for each of the left foot and right foot, that is, a total of 14 locations on the sole surface of the user's foot will be described as an example. In the example, the force or pressure on the sole surface is measured. First, a calculation is performed in which the measured values at each time indicated by the 14 time-series data illustrated in FIG. 19 are added. When such a calculation is performed, for example, the following calculation result can be obtained.

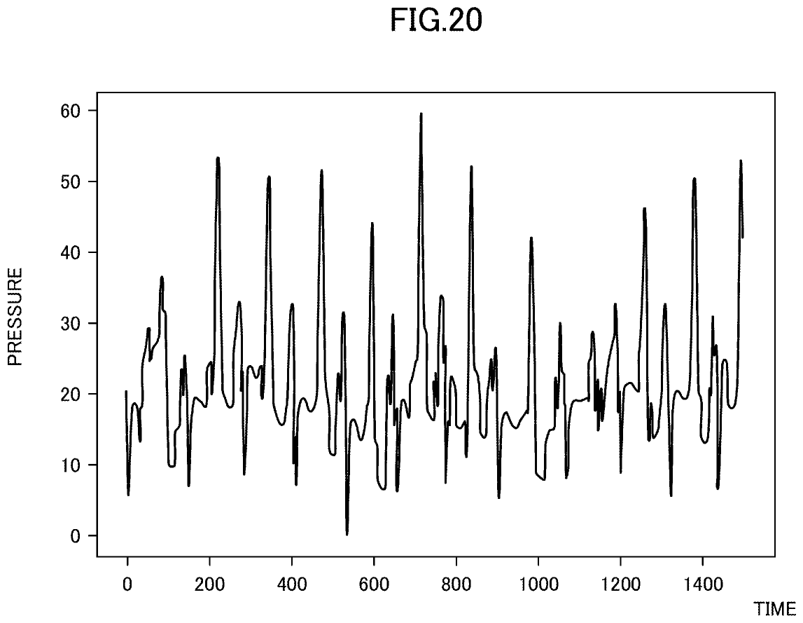

FIG. 20 is a diagram illustrating an example of the addition result. As illustrated in FIG. 20, by adding all 14 values of the measured values indicated by the time-series data at each time point, the value at each time point indicated by the addition result is calculated. When the FFT is performed on the calculation result, for example, the following FFT result is acquired.

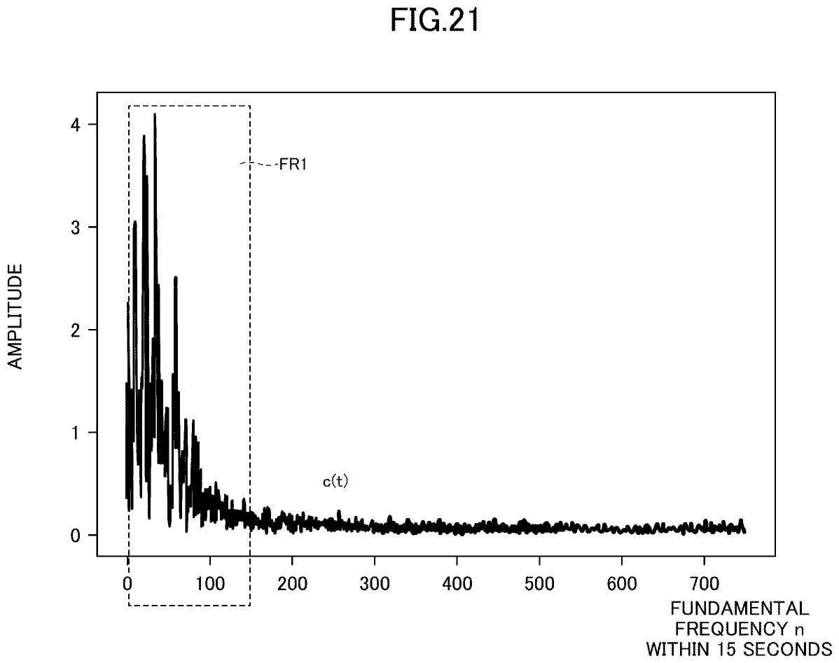

FIG. 21 is a diagram illustrating an example of the FFT result. For example, if the processing of FFT is performed on the calculation result as illustrated in FIG. 20, the FFT result as illustrated is acquired. Then, the following parameters can be acquired from the FFT result.

The “energy” is, for example, the value calculated by Formula (3) below (variable “E” in Formula (3)). Further, the “energy” is an example of the “energy” of the “FFT features” in FIG. 6.

$$\begin{matrix}[ Formula ( 3 ) ] & \\ \begin{matrix}E = \frac{\sum {\{ c ( t ) \}}^{2}}{\frac{N}{2}} & \end{matrix} & ( 3 )\end{matrix}$$

$$E represents energy .$$

$$c ( t ) is function of time t representing frequency spectrum .$$

$$N is the number of time points in one window , that is , the size of the window .$$

$$There are 1500 time points in a window of 100 Hz , 15 seconds , therefore , N = 1500$$

The “weighted average value of frequencies” is, for example, the value calculated by Formula (4) below (variable “WA” in Formula (4)). Further, the “weighted average value of frequencies” is an example of the “weighted average value of frequencies” of the “FFT features” in FIG. 6.

$$\begin{matrix}[ Formula ( 4 ) ] & \\ WA = \frac{\sum \{ c ( t ) \times t \}}{\sum \{ c ( t ) \}} & ( 4 )\end{matrix}$$

$$WA represents weighted average .$$

$$c ( t ) is function of time t representing frequency spectrum .$$

The “FFT feature” may be, for example, a skewness of the spectrum at a fundamental frequency from 0 Hz to 10 Hz (hereinafter simply referred to as “skewness”), which is calculated as follows.

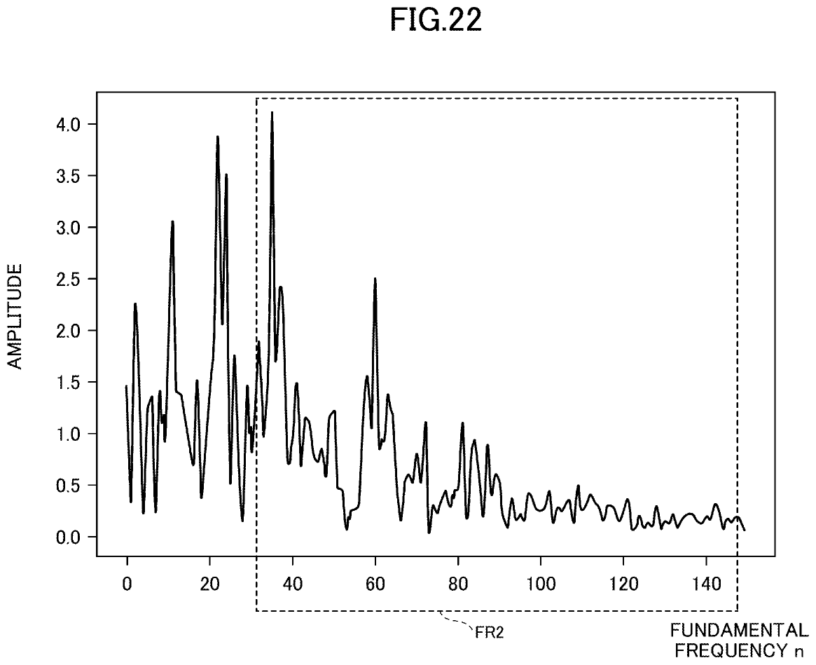

FIG. 22 is a diagram illustrating an extraction example of a fundamental frequency of 0 Hz to 150 Hz. The fundamental frequency from 0 to 150 Hz in FIG. 22 is the result of extracting the fundamental frequencies from 0 Hz to 150 Hz (hereinafter referred to as “first frequency band FR1”) out of the entire frequencies illustrated in FIG. 21. For such extraction results, the skewness can be calculated by the following Formula (5).

$$\begin{matrix}[ Formula ( 5 ) ] & \\ {g}_{i} = \frac{{m}_{3}}{{m}_{2}^{3 / 2}} , {m}_{i} = \frac{1}{n} {\sum }_{t = 1}^{n} {( c ( t ) - \overline{c} )}^{i} , n = 150 & ( 5 )\end{matrix}$$

$$n is fundamental frequency within 15 seconds .$$

$$c ( t ) is function of time t representing frequency spectrum .$$

$$\overline{c} represents average value of c ( t ) .$$

$$Frequency f is \frac{n}{15} .$$

$${g}_{1} is skewness .$$

$${m}_{2} is second cumulant .$$

$${m}_{3} is third cumulant .$$

$$i is coefficient .$$

When the coefficient “i” in Formula (5) is replaced by a secondary cumulant “m₂” with “i=2” and a tertiary cumulant “m₃” with “i=3”, the following Formula (6) is obtained.

$$\begin{matrix}[ Formula ( 6 ) ] & \\ skewness = \frac{\frac{1}{n} {\sum }_{t = 1}^{n} {( c ( t ) - \overline{c} )}^{3}}{{( \frac{1}{n} {\sum }_{t = 1}^{n} {( c ( t ) - \overline{c} )}^{2} )}^{3 / 2}} , n = 150 & ( 6 )\end{matrix}$$

In Formulas (5) and (6), “n” may be any value other than “150” depending on the setting or the like.

The skewness is an example of the “spectral skewness from 0 to 10 Hz” of “FFT features” in FIG. 6. Further, according to the relationship between the fundamental frequency and the frequency represented in Formula (5), the fundamental frequency from 0 Hz to 150 Hz (which is “n” in Formula (5)) is the frequency from 0 Hz to 10 Hz (which is “f” in Formula (5)).

Actions by humans are often performed at frequencies up to 10 Hz. Therefore, the frequency from 0 Hz to 10 Hz is preferably extracted.

Additionally, the “FFT feature” may be, for example, “the average value of the 2 Hz to 10 Hz spectrum” and “the standard deviation of the 2 Hz to 10 Hz spectrum” as calculated below, and the like. In order to calculate these values, first, a process of extracting frequency of 2 Hz to 10 Hz is performed with respect to the extraction result illustrated in FIG. 22. Specifically, the fundamental frequency of 30 Hz to 150 Hz in FIG. 22 (hereinafter referred to as “second frequency band FR2”) is extracted. The extraction result is as follows, for example.

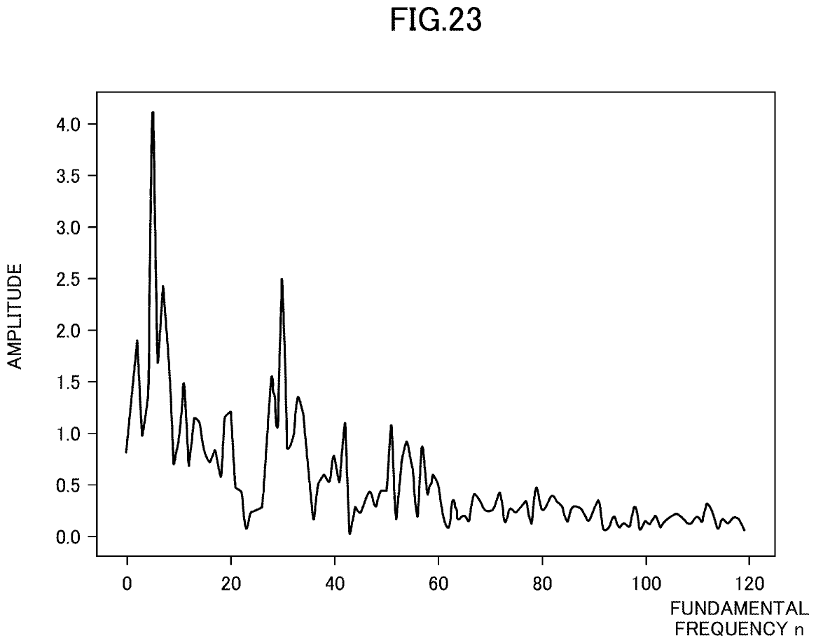

FIG. 23 is a diagram illustrating an extraction example of fundamental frequency of 30 to 150 Hz. That is, FIG. 23 is the extraction result of the fundamental frequency from 30 Hz to 150 Hz out of the entire frequency illustrated in FIG. 22.

A frequency of 2 Hz or less is a frequency considered to be a walking cycle. Accordingly, the frequency of 2 Hz or less is preferably eliminated because of overlap with the peak feature. Therefore, as illustrated, the fundamental frequency from 30 Hz to 150 Hz (i.e., 2 Hz to 10 Hz in frequency, according to the relationship between the fundamental frequency and frequency represented in Formula (5)) is preferably extracted.

The average value of the spectrum is then calculated based on the extraction result of the fundamental frequency from 30 Hz to 150 Hz. This calculation results in an example of the “average value of the 2 to 10 Hz spectrum” of the “FFT features” in FIG. 6.

Further, the standard deviation of the spectrum is calculated based on the extraction result of the fundamental frequency from 30 Hz to 150 Hz. This calculation results in an example of the “standard deviation of the 2-10 Hz spectrum” of the “FFT features” in FIG. 6.

Other statistics may be further calculated.

<Example of Filter>

A bandpass filter, a butterworth filter, or a low pass filter are preferably applied to the measurement data to be determined. In particular, the butterworth filter is preferable. The filtering process is preferably performed after step S3 and before step S4, for example, to apply to the measurement data. Specifically, the measurement data is as follows by filtering.

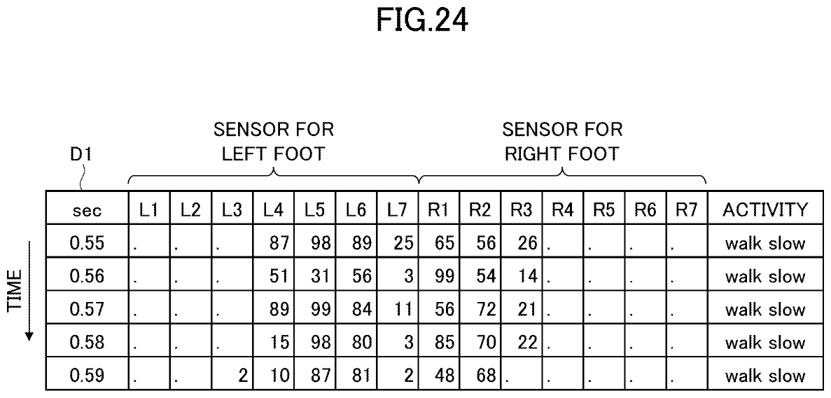

FIG. 24 is a diagram illustrating an example of measurement data before the filtering. For example, as illustrated in FIG. 7, seven sensors are installed for each of the left foot and right foot to measure the force or pressure at the sole surface of the user's foot in a total of 14 locations.

Therefore, the illustrated measured data is so-called raw data (hereinafter referred to as “pre-filter data D1”).

Then, filter processing for attenuating a frequency of 5 Hz or higher included in the measurement data is performed for the pre-filter data D1.

Due to the movement features of the legs, it is difficult for humans to move faster than 5 Hz. Accordingly, data including the frequency of 5 Hz or higher is likely to be noise indicating a movement other than the movement that can be performed by a human. Therefore, when the filter to attenuate the frequency of 5 Hz or higher is applied, the noise included in the measurement data can be reduced.

Accordingly, for example, a butterworth filter or the like that cuts off 10 Hz or less is preferably used in consideration of a margin or the like.

Further, in the illustrated example, the values are normalized to the pre-filter data D1 so that each value indicated by the measurement data is represented as a numerical value within a predetermined range. The result of such a process is as follows.

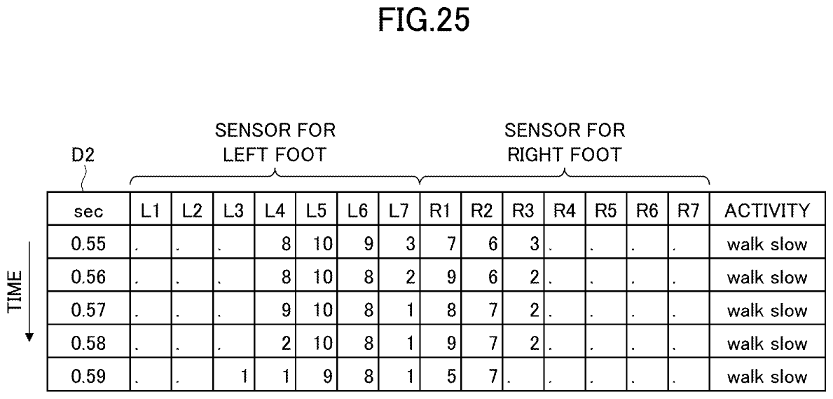

FIG. 25 is a diagram illustrating an example of the measurement data after the filtering. The illustrated example is data illustrating an example of the result of applying the butterworth filter to the pre-filter data D1 (hereinafter referred to as “post-filter data D2”).

That is, the post-filter data D2 is the data in which the noise included in the measurement data acquired in step S3 is attenuated. When such data becomes the data to be determined, the behavior determination apparatus can accurately determine the behavior of the user.

<Function Configuration Example>

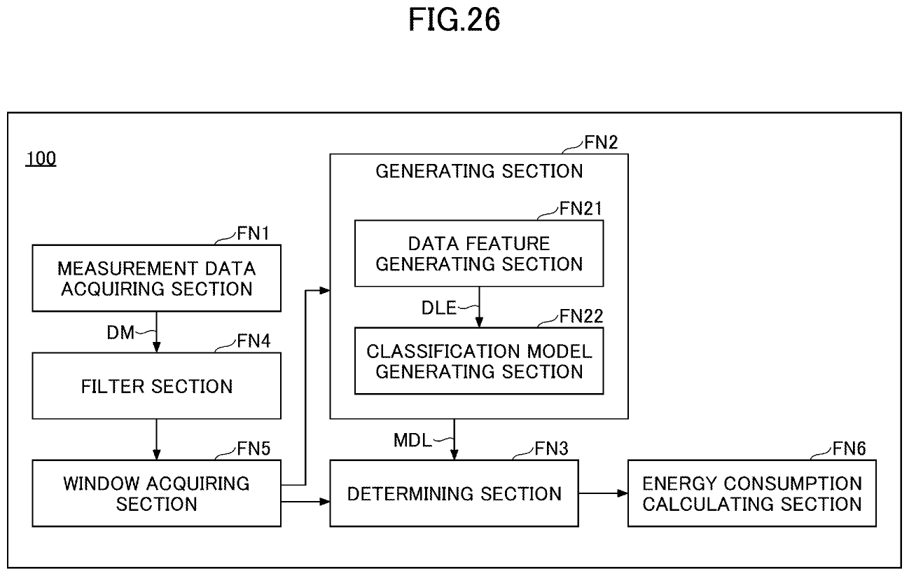

FIG. 26 is a functional block diagram illustrating a functional configuration example of a behavior determination system. For example, the behavior determination system 100 has a functional configuration including a measurement data acquiring section FN1, a generating section FN2, and a determining section FN3. Further, as illustrated in FIG. 26, the behavior determination system 100 preferably has a functional configuration that further includes a filter section FN4, a window acquiring section FN5, and an energy consumption calculating section FN6. Hereinafter, the functional configuration illustrated in FIG. 26 will be described as an example.

The measurement data acquiring section FN1 performs a measurement data acquisition procedure in which measurement data DM indicating the pressure or force measured by one or more sensors installed on the sole surface of the user's foot is acquired. For example, the measurement data acquiring section FN1 is implemented by the connection I/F 205.

The generating section FN2 performs a generation procedure that generates a classification model that classifies the behavior of the user by using the measurement data DM, the data feature acquired from the measurement data DM, and the like as training data DLE in machine learning. For example, the generating section FN2 is implemented by the CPU 201 or the like.

As illustrated in FIG. 26, the generating section FN2 preferably has a configuration having a data feature generating section FN21, a classification model generating section FN22, or the like.

The data feature generating section FN21 generates a data feature or the like to generate the training data DLE.

The classification model generating section FN 22 generates a classification model MDL based on the learning process of the training data DLE.

The determining section FN3 performs a determination process in which a behavior of the user is determined using the classification model MDL based on the measurement data DM. For example, the determining section FN3 is implemented by the CPU 201 or the like.

The filter section FN4 performs filtering to apply, for example, a butterworth filter or a low-pass filter to the measurement data DM to attenuate a frequency of 5 Hz or higher. For example, the filter section FN4 is implemented by the CPU 201 or the like.

The window acquiring section FN5 performs a window acquisition process in which a window that determines a range to be used for determination by the determining section FN3 is set with respect to the measurement data DM and is slid on the time axis to set the window. For example, the window acquiring section FN5 is implemented by the CPU 201 or the like.

The energy consumption calculating section FN6 performs a process for calculating the energy consumption for allocating each energy consumption with respect to the behavior and calculating the total energy consumption of the user by adding up the energy consumption. For example, the energy consumption calculating section FN6 is implemented by the CPU 201 or the like.

Further, the behavior determination system 100 may have the following functional configuration.

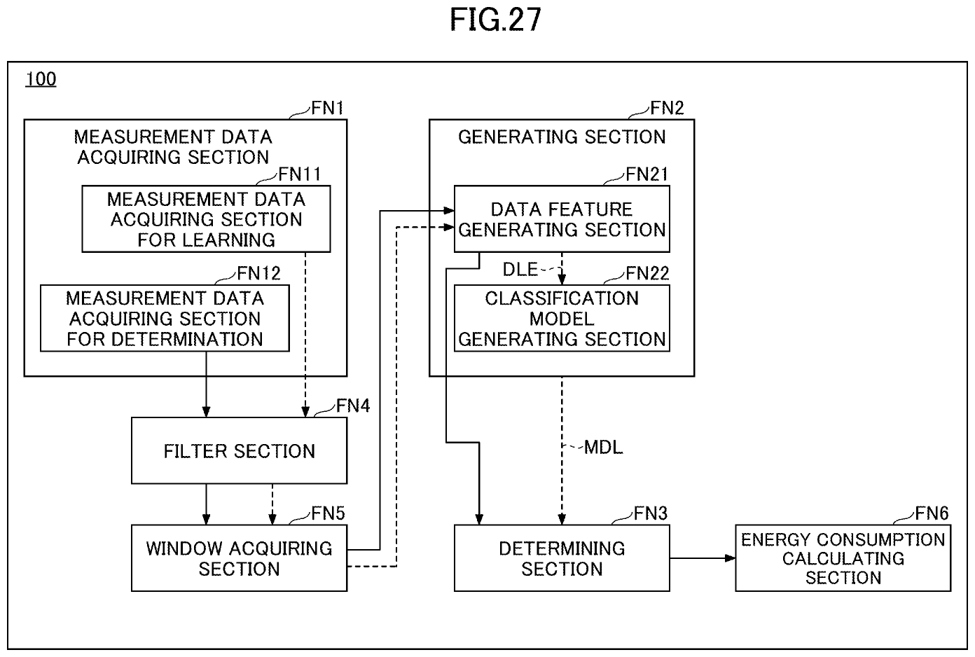

FIG. 27 is a functional block diagram illustrating a modification of the functional configuration of a behavior determination system. As illustrated, the measurement data acquiring section FN1 may have a functional configuration that includes a measurement data acquiring section for learning FN11 and a measurement data acquiring section for determination FN12.

The measurement data acquiring section for learning FN 11 acquires measurement data that is used to generate a classification model MDL. In FIG. 27, the main data flow in a learning process is represented by “dashed lines”.

The measurement data acquiring section for determination FN12 acquires the measurement data to be determined for behavior. In FIG. 27, the main data flow in a determination process is represented by “solid lines”.

The functional configuration is not limited to the configuration illustrated in the figure. For example, a data feature generating section FN21 and a determining section FN3 may be integrated. Further, a filter section FN4, a window acquiring section FN5, and the data feature generating section FN21 may be integrated. Further, the filter section FN4, the window acquiring section FN5, the data feature generating section FN21, and the determining section FN3 may be integrated.

When the above-described functional configuration is used, for example, processes can be performed as follows.

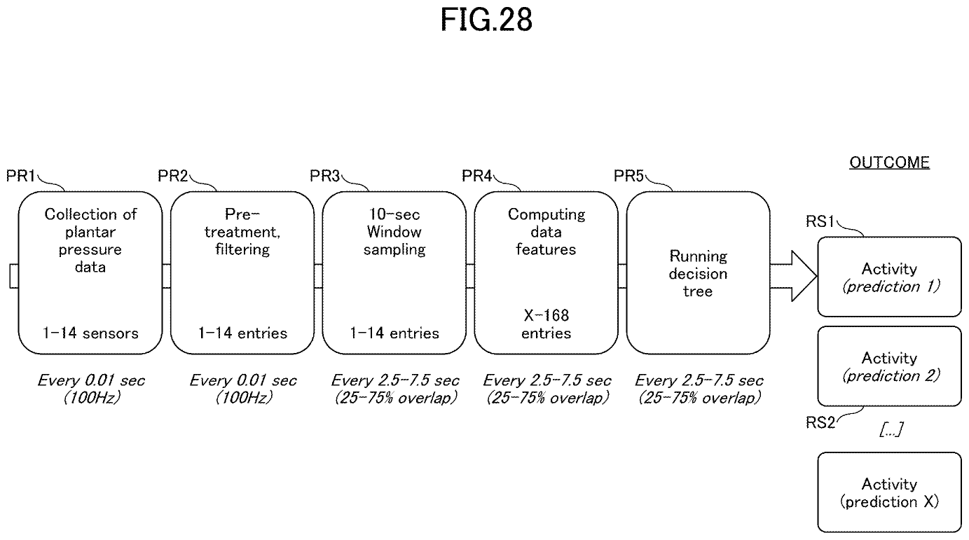

FIG. 28 is a diagram illustrating an example of a determination process of arbitrary measurement data by a behavior determination system. First, as illustrated in FIG. 26, before performing the processing, the measurement data for generating the training data DLE is acquired by the measurement data acquiring section FN1. By the measurement data DM acquired in this manner, the generating section FN2 generates the data feature to generate the training data DLE. Further, the generating section FN2 performs a process of generating the classification model MDL, that is, a learning process. Then, the classification model MDL is generated.

In this way, the classification model MDL is generated in advance by the learning process, and the determination process is processed in order from a measurement data acquisition procedure PR1.

In the measurement data acquisition procedure PR1, the behavior determination system acquires the measurement data DM.

In a filter procedure PR2, the behavior determination system applies a filter to the measurement data DM.

In a window acquisition procedure PR3, the behavior determination system sets a window with respect to the measurement data DM or the like to which the filter is applied. Next, the behavior determination system performs an extraction procedure PR4, in which a parameter or the like is extracted from the range where the window is set. Then, a determination procedure PR5 is performed using the range specified in the window and the extracted parameter.

In the determination procedure PR5, the behavior determination system determines behavior by using the classification model MDL generated by the learning process or the like. Specifically, the behavior is set to be classified in advance as illustrated in FIG. 12, FIG. 13, or the like, by the classification model MDL.

Then, according to the classification model MDL, a behavior is determined for each window based on the measurement data and the data feature (a parameter or the like) acquired from the measurement data. The determined behavior is, for example, as illustrated, a first determination result RS1, a second determination result RS2, or the like.

In this manner, it is preferable that a process using the determination result is performed by using the data such as the first determination result RS1 and the second determination result RS2 to be determined, as in the case of the energy consumption calculating section FN6. The process using the determination result is not limited to the energy consumption calculation.

<Example of Using Voting>

The behavior determination system may also determine a behavior at a predetermined time intervals (hereinafter, “voting” means outputting a result of determining the behavior by a process using a classification model at a predetermined time interval) and output the determination result in which a single behavior is ultimately determined by using a plurality of voting results. For example, a predetermined amount of time, which is a unit of time for voting, may be set to approximately several seconds in advance.

The predetermined time interval may be set to the size of the window. That is, a vote is a determination made in units of time shorter than the final determination. Specifically, if a final determination is made in units of about “30” to “60” seconds, a vote may be made in units of, for example, “2.5” to “7.5” seconds. In this manner, a plurality of voting results are obtained before the final determination is made.

The behavior determination system then makes a final determination based on the plurality of voting results. For example, the behavior determination system adopts the behavior of the most frequent voting result of the plurality of voting results as the final determination result.

For example, three voting results of “walking”, “running”, and “walking” are assumed to be acquired. In this example, there are two voting results of “walking”, which is the most frequent voting result of the plurality of voting results. Therefore, the behavior determination system makes a final determination that the behavior of the user at the time when the three voting results are acquired is “walking”, and outputs the determination result indicating “walking” to the user.

For example, in the determination process illustrated in FIG. 28, first, the first determination result RS1 is output with respect to a certain window of 10 seconds. Next, assuming that the overlap is 50%, after five seconds, the second determination result RS2 is output with respect to a certain window of 10 seconds by the same determination process. In this way, by the determination process, “X” determination results are output, such as, “first determination result RS1” to “Xth determination result”.

Then, each determination result from the first determination result to the Xth determination result is regarded as “voting”. Next, the most frequent voting result of the voting results is calculated from the start to 60 seconds later. In this way, the determination result with the most frequent voting result may be adopted as the final determination result of “60 seconds”.

Thus, when the determination is made based on the plurality of voting results, the behavior determination system can determine the behavior with high accuracy.

<First Experimental Results>

With the above-described configuration, for example, a behavior can be determined with high accuracy as follows.

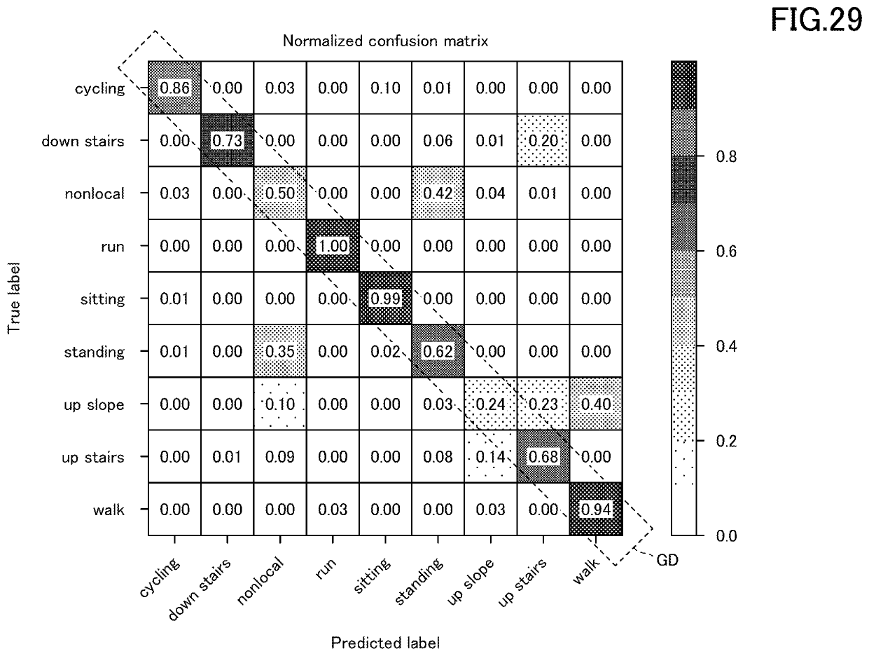

FIG. 29 is a diagram illustrating the experimental results. The experimental results illustrated in FIG. 29 are the results of verifying whether the determination result of the behavior determination system, which is the functional structure illustrated in FIG. 26, classifying the behavior, is consistent with the actual behavior and evaluating the so-called “correct answer rate”. At this time, the measurement data is the data measured when 14 people acted according to 11 behavioral patterns for four minutes. The window is acquired every 5 seconds, and a single window holds ten seconds of the measurement data. In addition, a random forest was used for the classification model. The calculation limit was set to “100” for the number of decision trees, “2” for the minimum number of samples required to allow branching (i.e., Minimum Sample Split), “1” for the Verbose, “−1” for the Number of jobs, “25” for Random state. The numerical value in the figure indicates a ratio, for example, “1.00” indicates “100%”.

Specifically, the horizontal axis (i.e., “predicated label”) is the behavior predicted by the behavior determination system, that is, the determination result. On the other hand, the vertical axis (i.e., “true label”) is an actually taken behavior (hereinafter referred to as “actual behavior”).

Therefore, it can be evaluated that the higher the ratio of the determination result illustrated on the horizontal axis to the actual behavior illustrated on the vertical axis, the more accurate the behavior was determined. In FIG. 29, the experimental result illustrated on the diagonal line is the case where the determination result and the actual behavior match. Hereinafter, as illustrated, an experimental result illustrated on the diagonal lines is referred to as “correct answer GD”.

The accuracy as a whole, that is, the ratio of the correct answers GD is “84%”, and the behavior can be determined with high accuracy as a whole. In particular, the behavior determination system can determine behaviors such as running, sitting, walking, and riding a bicycle with high accuracy of 80% or more, as illustrated in FIG. 29.

In particular, each behavior of running, sitting, and walking can be determined with an accuracy of 90% or more, and such a highly accurate determination is difficult in the above-mentioned Patent Document 2 and the like.

<Second Experimental Results>

In addition, it is preferable to use a Support Vector Machine (SVM) or a decision tree as the classification model as follows.

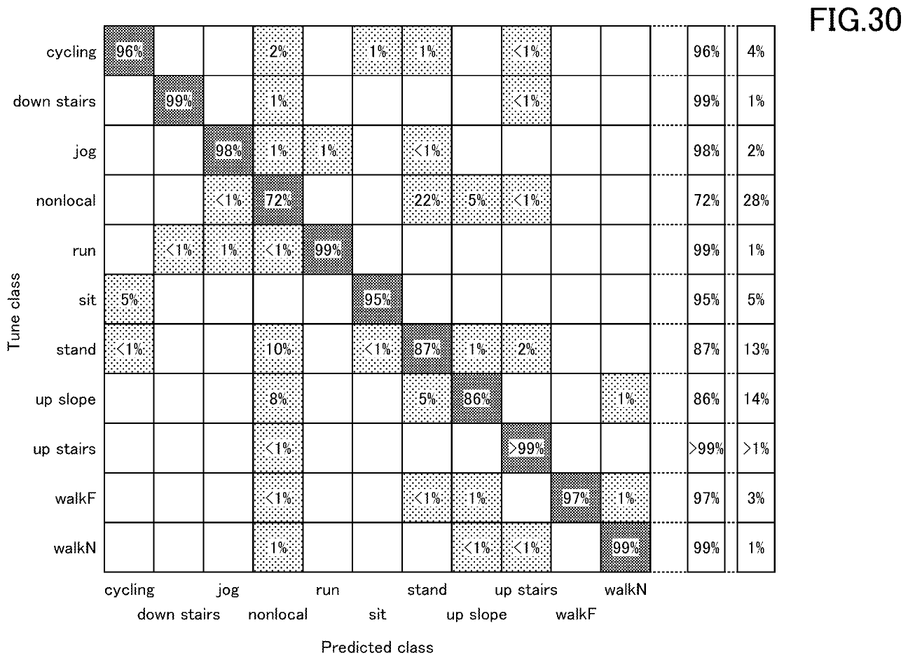

FIG. 30 is a diagram illustrating the experimental results using the SVM classification model. Similar to FIG. 29, the horizontal axis and the vertical axis represent the determination result and actual behavior. Accordingly, similarly to FIG. 29, the experimental results illustrated on the diagonal line are “correct answers” in which the determination result and the actual behavior match.

The accuracy as a whole, that is, the ratio of the correct answers is “92.6%”, and the behavior can be determined with high accuracy as a whole.

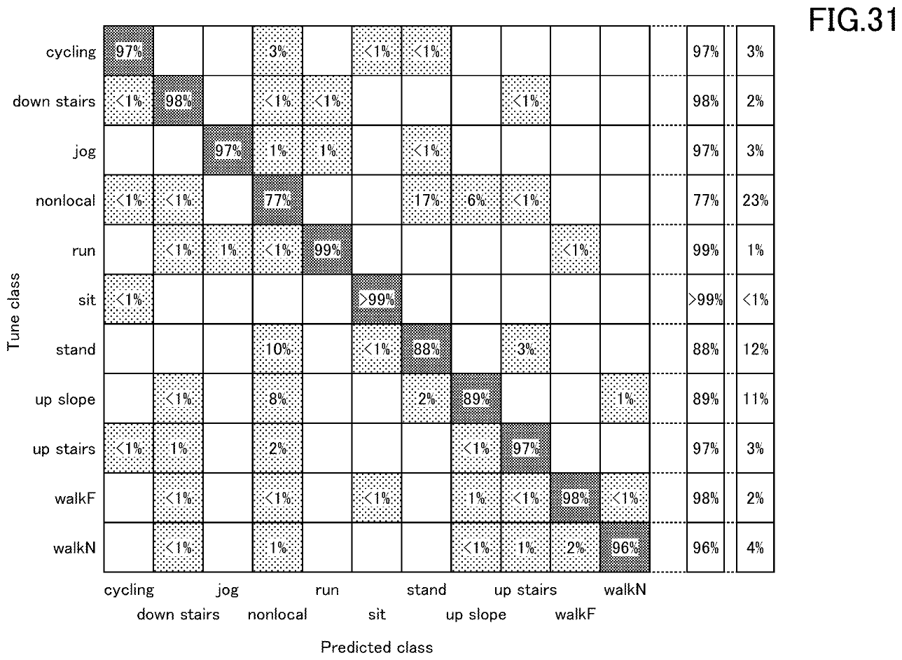

FIG. 31 is a diagram illustrating the experimental results using the classification model of the decision tree. Similar to FIG. 29, the horizontal axis and the vertical axis represent the determination result and actual behavior. Accordingly, similarly to FIG. 29, the experimental results illustrated on the diagonal line are “correct answers” in which the determination result and the actual behavior match. FIG. 31 illustrates experimental results when the same measurement data as in FIG. 30 is used and the classification model used is changed.

The accuracy as a whole, that is, the ratio of the correct answers is “93.7%”, and the behavior can be determined with high accuracy as a whole. In addition, as can be seen from FIG. 30, it is possible to determine the behavior more accurately by using the decision tree.

The use of such a behavior determination system enables to make a determine with regard to a non-uniform action, with high accuracy (a high accuracy that is difficult to achieve with Patent Document 2 can be achieved).

As described above, the classification model is not limited to the SVM or the decision tree. That is, the behavior determination system may be configured to apply so-called Artificial Intelligence (AI), in which machine learning is performed to learn the determination method.

In the above description, pressure is mainly described as an example, but the force may be measured by using a force sensor. Further, a pressure or the like that can be calculated by measuring the force and dividing the force by the area may be used in a state where the area for measuring the force is known in advance.

The behavior determination system 100 is not limited to the system configuration illustrated in the drawings. That is, the behavior determination system 100 may further include an information processing device other than the one illustrated in the drawings. On the other hand, the behavior determination system 100 may be implemented by one or more information processing devices, and may be implemented by less information processing devices than the illustrated information processing devices.

Each device does not necessarily have to be formed by one device. In other words, each device may be formed by a plurality of devices. For example, each device in the behavior determination system 100 may perform each process by a distributed processing, a parallel processing, or redundant processing executed by the plurality of devices.

All or a portion of each process according to the embodiments and modifications may be described in a low-level language, such as an assembler or the like, or a high-level language, such as an object-oriented language or the like, and may be performed by executing a program that causes the computer to perform a behavior determination method. In other words, the program may be a computer program for causing the computer, such as the information processing system or the like including the information processing device or the plurality of information processing devices, to execute each process.

Accordingly, when the behavior determination method is executed based on the program, the arithmetic unit and the control unit of the computer perform calculations and control based on the program for executing each process. The storage device of the computer stores the data used for the processing, based on the program, in order to execute each process.

The program may be stored and distributed on a non-transitory computer-readable storage medium. The non-transitory computer-readable storage medium includes a medium such as an auxiliary storage device, a magnetic tape, a flash memory, an optical disk, a magneto-optical disk, a magnetic disk, or the like. In addition, the program may be distributed over a telecommunication line.

Although the preferred embodiments of the present invention are described above in detail, the present invention is not limited to the embodiments described above, and various modifications, variations, and substitutions may be made within the scope of the present invention.

What is claimed is:

1. A behavior determination apparatus, comprising:
a classification model configured to classify a behavior of a user;
a measurement data receiving device configured to acquire measurement data indicating a pressure or a force measured by one or more sensors provided on a sole surface of a foot of the user;
a memory; and
a processor configured to calculate a data feature by performing data processing on the measurement data and determine the behavior of the user by using the classification model.

2. The behavior determination apparatus according to claim 1, wherein the processor is further configured to attenuate a frequency higher than a human activity frequency with respect to the measurement data.

3. The behavior determination apparatus according to claim 1, wherein at least one of the one or more sensors is provided at least at a widest width of the sole surface of the foot of the user in a direction orthogonal to a traveling direction of the user.

4. The behavior determination apparatus according to claim 1, wherein the processor is further configured to set a window, in the data processing, that defines a range to be used for calculating the data feature with respect to the measurement data, and
wherein the range is set by sliding the window along a time axis.

5. The behavior determination apparatus according to claim 1, wherein, with respect to the measurement data, at least one of a statistical feature, a peak feature, a walking cycle feature, an FFT feature, a sole pressure tendency feature, or a combination thereof is used as the data feature to generate the classification model and determine the behavior by using the classification model.

6. The behavior determination apparatus according to claim 1, wherein, with respect to the measurement data,
a distribution of either the pressure or the force in a traveling direction of the user and an orthogonal direction to the traveling direction,
a Pearson correlation coefficient of either the pressure or the force in the traveling direction and the orthogonal direction, or
a distribution of averaged values of either the pressure or the force in the traveling direction and the orthogonal direction is used as the data feature to generate the classification model and determine the behavior by using the classification model.

7. The behavior determination apparatus according to claim 1, wherein, with respect to the measurement data from the sensors at a same position on both left foot and right foot,
a time from a first time point to a second time point, the first time point at which the force or the pressure in a first foot becomes a value smaller than a threshold, the first foot being one of the left foot and the right foot, and the second time point at which the force or the pressure in a second foot starts to increase from a value smaller than the threshold, or
an average time from the first time point to the second time point is used as the data feature to generate the classification model and determine the behavior by using the classification model.

8. The behavior determination apparatus according to claim 1, wherein the data feature is determined based on a number and a location of the sensors.

9. The behavior determination apparatus according to claim 1, wherein the classification model is a decision tree, and the behavior is determined based on the data feature in the decision tree.

10. The behavior determination apparatus according to claim 9, wherein the classification model is a plurality of decision trees, and
the processor determines, based on determination results by the plurality of decision trees, a determination result with a largest number of determinations as the behavior of the user.

11. The behavior determination apparatus according to claim 1, wherein the processor is further configured to generate the classification model by using measurement data measured for a plurality of users as training data for machine learning.

12. The behavior determination apparatus according to claim 11, wherein the processor uses, as the training data, the data feature and a behavior label given based on the behavior at a time of the measurement data being acquired.

13. The behavior determination apparatus according to claim 1, wherein the classification model is configured to classify the behavior as sitting, standing, non-locomotive, walking slow, walking fast, walking on a slope, going up stairs, going down stairs, running slow, running fast, or riding on a bicycle.

14. The behavior determination apparatus according to claim 1, wherein voting is performed at a predetermined interval and the processor determines the behavior based on a most frequent voting result of a plurality of voting results.

15. A behavior determination system, comprising:
a classification model configured to classify a behavior of a user;
a measurement data receiving device configured to acquire measurement data indicating a pressure or a force measured by one or more sensors provided on a sole surface of a foot of the user;
a memory; and
a processor configured to calculate a data feature by performing data processing on the measurement data and determine the behavior of the user by using the classification model.

16. A behavior determination method to be implemented in a behavior determination apparatus that includes a classification model to be used for classifying a behavior of a user, the method comprising:
acquiring, by the behavior determination apparatus, measurement data indicating a pressure or a force measured by one or a plurality of sensors provided on a sole surface of the foot of the user;
calculating, by the behavior determination apparatus, a data feature by performing data processing on the measurement data; and
determining, by the behavior determination apparatus, the behavior of the user by using the classification model.

17. A non-transitory computer-readable storage medium having stored therein a program for causing a computer to execute the behavior determination method of claim 16.
